(19) United States
(12) Patent Application Publication
(10) Pub. No.: US 2010/0099189 A1
(43) Pub. Date: Apr. 22, 2010

(54) Methods for Rejuvenating Cells In Vitro and In Vivo

(76) Inventor: Jifan Hu, Palo Alto, CA (US)

Correspondence Address: THE LUTHER LAW FIRM, PLC, 10575 N 114TH STREET, SUITE 103, SCOTTSDALE, AZ, 85259, US

(21) Appl. No.: 12/090,247

(22) PCT Filed: Oct. 16, 2006
(86) PCT No.: PCT/US06/40723

§ 371 Date: Sep. 22, 2008

Related U.S. Application Data
(60) Provisional application No. 60/726,915, filed on Oct. 14, 2005.

(51) Int. Cl. C12N 5/071

(52) U.S. Cl. 435/383

(57) ABSTRACT

The present invention provides methods for rejuvenating cells, tissues and the whole body. Also provided are rejuvenating buffers and agents as well as kits for rejuvenating cells. Also provided are methods for dedifferentiating somatic cells and differentiating the cells into other cell types.

### CROSS REFERENCE TO RELATED APPLICATION

This application is a 35 U.S.C. 371 application based on PCT/US2006/040723, which was filed Oct. 16, 2006, and which claims the benefit of U.S. Provisional Patent Application Ser. No. 60/726,915, filed Oct. 14, 2005, and is a continuation-in-part of pending U.S. Application No. 11.358,465, which was filed on Feb. 21, 2006, the entire disclosures of which are incorporated herein by reference.

### STATEMENT REGARDING FEDERALLY SPONSORED RESEARCH OR DEVELOPMENT

This invention was made with United States government support awarded by the following agencies: DOD grant #W81XWH-04-1-0597 and NIH/NCI grant #1 R43CA 103553-01. The United States has certain rights in this invention.

### BACKGROUND

1. Technical Field

This invention is related to methods of rejuvenating cells and human clinical and veterinary uses of these rejuvenated cells, and more particularly to methods of rejuvenating somatic cells to become pluripotent or multipotent embryonic stem or stem-like cells. The rejuvenating method can also be applied to mammalian organs and bodies.

2. Background Art

Aging is an inevitable process of life. Aging is a syndrome of changes that are deleterious, progressive, universal and thus far irreversible. Cellular aging is characterized by the reduced functionality of the cell, declining ability to respond to stress, increasing homeostatic imbalance, and increased risk of disease. Thus, aging itself may be regarded as a “disease process” and is an accumulation of damage to macromolecules, cells, tissues and organs.

Cellular aging is regulated by biological clocks operating throughout the life span of the subject, depending on changes in gene expression that affect the systems responsible for maintenance, repair and defense responses. Aging is associated with the shortening of telomeres in the process of DNA replication during cell division. Aging is accelerated by cumulative mutations and damages, ranging from macromolecules (DNA, RNA, proteins, carbohydrates, and lipids) to tissues by free radicals, glycation, radiation, cross-linking agents, etc.

A number of gene pathways have been identified in the aging process. One of these pathways involves the gene Sir2, an NAD+-dependent histone deacetylase. Extra copies of Sir2 are capable of extending the lifespan of both worms and flies. The human analogue SIRT1 protein has been demonstrated to deacetylate p53, Ku70, specific histone residues, and the forkhead family of transcription factors. Superoxide dismutase, a protein that protects against the effects of mitochondrial free radicals, can extend yeast lifespan in stationary phase when it is overexpressed. It is not known, however, whether these mechanisms also exist in humans since there are obvious differences in biology and pathophysiology between humans and model organisms.

The quality of life usually is reduced with aging. Because of aging, collagen and elastin in tendons and ligaments become less resilient and more fragmented, particularly due to glycation (cross-linking of proteins by sugar). Articular cartilage becomes frayed and the synovial fluid between joints becomes “thinner”. Decline in circulatory function contributes to this process. Collagen and elastin also cross-link in skin, resulting in a loss of elasticity. The protein keratin in fingernails is also a component of the outer layer of skin (epidermis), which provides “water-proofing.” The epidermis thins with age, leading to wrinkles Decreased secretion by sweat glands increases vulnerability to heat stroke. When melanocytes (cells that produce the skin and hair-coloring substance melanin) associated with hair follicles cease functioning, hair turns white. Partial reduction of melanocyte function results in hair that appears gray. Yet 90% of Caucasians show increased melanin in the form of brownish spots on the back of their hands (“liver spots”). Loss of flexibility of the proteins collagen and elastin in the lung results in less elastic recoil. Ventilation becomes more difficult, which reduces air exchange and respiration and thus the capacity to perform work.

Animal cells can be classified as germ cells (sperm or egg), stem cells, and somatic cells (differentiated functioning body cells). Embryonic fibroblasts in tissue culture cease dividing before they reach the Hayflick Limit of 50 divisions (Hayflick L, Moorhead P S Exp Cell Res 1961, 25:585-621). Germ cells, stem cells and “immortalized” cancer cells contain an enzyme called telomerase that replaces lost telomeres, thus preventing them from experiencing the Hayflick Limit. In human germ cells, and approximately 85% of cancer cells, the enzyme human TElomerase Reverse Transcriptase (hTERT) and an RNA template are sufficient to create new telomeres. Defects in proteins required to maintain telomere function can also lead to chromosome instability and cancer. Telomerase expression also makes cells more resistant to apoptosis induced by oxidative stress.

Human somatic cells that have been transfected with a reverse transcriptase subunit of telomerase express telomerase. Such cells exhibited 20 population doublings beyond their Hayflick Limit and continued to exhibit normal, healthy and youthful cellular appearance and activity. Such results create a realistic hope that preservation of youth in some tissues by a form of gene therapy that either induces the expression of native telomerase in somatic cells or adds genetic material to cells consisting of an engineered telomerase superior to the natural form may be possible.

Extensive studies have been conducted on ways to slow the aging processes to extend average lifespan through lifestyle changes and preventative disease prophylaxis (e.g., reducing calorie intake while maintaining adequate nutrition, eating low-fat/high-fiber diets, avoiding tobacco and alcohol, exercising, and taking antioxidant supplements. However, without extreme lifestyle changes, it is difficult to gain much immediate benefit to slow aging.

By definition, stem cells are undifferentiated cells, which are able to self-renew and differentiate into various functional, mature cells ranging from neuronal cells to muscle cells. Undifferentiated cells divide to form a daughter cell which differentiates to a specific somatic cell and a stem cell. Embryonic stem cells (hereinafter “ESCs”) are derived from an embryo and are pluripotent in nature. Pluripotent cells can give rise to most but not all the tissues necessary for fetal development. Pluripotent cells specialize into multipotent cells that commonly give rise to cells with a particular function (e.g., multipotent blood stem cells produce red cells, white blood cells and platelets). ESCs are able to differentiate into a particular cell, tissue or even an organ type depending on the differentiating conditions used. Human ESCs are useful in cell replacement therapies and implantation to treat diseases, such as Parkinson's disease, tissue grafting and screens for drugs and toxins. ESCs can also be used in the development of cell cultures for transplantation and manufacture of bio-pharmaceutical products, such as insulin, antibodies, and factor VIII.

ESCs hold promise in curing many human diseases. However, there are several concerns over ESCs. First, there are ethical and political issues regarding obtaining ESCs from fetuses. Second, research projects funded by NIH are restricted to a limited set of 22 ESC lines, which may be not enough for basic research studies. Third, the immune system in our body protects against the implanted ESCs. As a result, it may cause rejection of the implanted ESCs or cord blood stem cells and lead to graft-versus-host disease.

In addition to using nuclear transfer, conversion of somatic cells into pluripotent cells by cell reprogramming has been attempted. Hansis et al (Curr Biol 2004, 14:1475-1480) described a method to reprogram somatic cells, including human lymphocytes and human 293T kidney cells, with Xenopus egg extracts. They found that BRG1 was required for the in vitro reprogramming. However, it is not clear whether the cells gained pluripotent properties. Tada et al (Curr Biol 2001, 11:1553-1558) described a protocol to transform somatic cells into pluripotent cells by in vitro cell hybridization. They fused terminally differentiated thymocytes with embryonic stem cells (ESCs). These ESC-thymocyte hybrid cells had the pluripotency of the original ESCs. However, these hybrid cells were tetraploid cells with unstable genomes and can not be directly used for clinical therapies. Do and colleagues (Stem Cells 2004, 22:941-949) described a similar protocol to transform somatic cells into pluripotent cells by in vitro cell hybridization. They fused ESCs with neurosphere cells (NSCs). The fused cells had activated Oct4, a gene essential for pluripotency in ESCs. They further showed that the reprogramming capacity of ESCs was derived from the ESC nuclei. Similarly, these hybrid cells were tetraploid cells and cannot be used in cell replacement therapy. Collas and his colleagues (Philos Trans R Soc Lond B Biol Sci. 2003, 358:1389-1395) have published a series of papers to transdifferentiate cells for cell therapy. However, due to technical difficulties they have not yet reported the successful transdedifferentiation of somatic cells into pluripotent cells.

While it seems unlikely aging could be stopped at a youthful age, replacing or repairing damaged organs, tissues, cells and even molecules appears to be a more robust strategy. These strategies can rejuvenate cells and restore function to aged organisms. It is thus the objective of the present invention to overcome or at least alleviate some of the problems of the prior art and to provide a more effective and practical method for efficiently rejuvenating cells in vitro and in vivo, and the following disclosure provides a practical system which meets the needs in the art as described above and provides additional advantages as well.

### BRIEF SUMMARY OF THE INVENTION

It is an object of the present invention to provide technically straight-forward methods of rejuvenating cells, tissues and whole bodies.

In one embodiment, there is provided a method for rejuvenating aged cells with the steps of a. providing a sample including aged somatic cells; b. providing a rejuvenating solution including rejuvenating extract, albumin, ATP, phosphocreatine, creatine kinase, RNase inhibitor and nucleotide phosphates; c. combining the aged cells with the rejuvenating solution and incubating for a sufficient time for the constituents of the rejuvenating solution to penetrate the cells; and d. adding a solution of appropriate cell medium such as KO-DMEM with calcium chloride and optionally antibiotics. In this method, the rejuvenating extract can be extracted from cells at an early stage of development selected from egg, fertilized egg, blastocysts, embryo, cord blood stem cell, stem cell, primordial germ cell, embryonic stem cell or fetus. The fetal cells can be fetal liver cells. Optionally, the rejuvenating extract is extracted from cells or portions of cells comprising nuclei. Optionally, the rejuvenating extract can be obtained from cells or nuclei at any phase of the cell cycle or from a plurality of cells or nuclei at a variety of phases of the cell cycle.

In another embodiment, there is provided a method of rejuvenating cells into pluripotent embryonic stem-like (ESL) cells with the steps of providing a sample including aged somatic cells; providing a rejuvenating solution including rejuvenating extract, albumin, ATP, phosphocreatine, creatine, RNase inhibitor and nucleotide phosphates; combining the aged cells with the rejuvenating solution and incubating for a sufficient time for the constituents of the rejuvenating solution to penetrate the cells to produce rejuvenated cells; adding to the rejuvenated cells a solution of cell medium, calcium chloride and optionally antibiotics to expand the cell population; growing the expanded cells in inverted hanging droplets on the cover of the plate or an uncoated Petri dish for a sufficient time to accelerate cell aggregation; growing the cell aggregations in suspension to form embryoid bodies (EBs) on a dilute agarose gel or on coated plates and disks, or on Matrigel in appropriate medium supplemented with growth factors; culturing embryoid body-like cells on top of feeder cells; and selecting cell colonies with the same morphology as stem cells, whereby the somatic cells are dedifferentiated into pluripotent cells for cell therapy and cosmetic applications. The dilute agarose gel can be about 0.2% to 2% agarose.

In another embodiment, there is provided a method of rejuvenating somatic cells into pluripotent ESL cells by transfection with embryonic stem cell (ESC) mRNA that has the steps of extracting mRNA from a pluripotent cell; encapsulating the mRNA in at least one liposome delivery reagent; exposing the somatic cells to the liposomes of mRNA; growing the exposed somatic cells in inverted hanging droplets on the cover of a plate or uncoated Petri dish for a sufficient time to accelerate cell aggregation; growing the aggregated cells in suspension to form EBs on top of dilute agarose gel or in an uncoated Petri dish; culturing the EB-like cells on feeder cells, coated plates and disks or on Matrigel in appropriate media supplemented with growth factors; and selecting cell colonies with a morphology of stem cells, whereby somatic cells are dedifferentiated into pluripotent cells for cell therapy and cosmetic applications. The pluripotent cells can be ESCs, primordial germ cells (PGCs), fetal cells, eggs, fertilized eggs, umbilical cord blood stem cells, tissue stem cells, blastocysts cells or a combination thereof. The sufficient time is about two hours to overnight. Instead of transfecting by liposomes, the cells can be subjected to electroporation or viral-mediation.

In another embodiment, there is provided a method of forming pluripotent, diploid ESL cells for autologous transplantation by cell fusion of ESCs with mammalian somatic cells, with the steps of a. providing ESCs; b. disabling DNA replication in the ESCs; c. mixing the non-replicating ESCs with a plurality of somatic cells; d. fusing the non-replicating ESCs and the somatic cells by adding polyethylene glycol thereto; e. growing the fused cells in inverted droplets to produce aggregated cells; f. growing the aggregated cells in suspension on dilute agarose gel to produce EBs for 1-10 passages; g. growing the EBs on feeder cells, coated plates and disks, or Matrigel in appropriate medium supplemented with growth factors; and h. selecting cell colonies of patient-specific ESL cells that have the morphology typical of stem cells and comprise mammalian-specific diploid ESL cells. The ESC may be replaced with stem cells and the somatic cells can be fibroblasts. The step of disabling DNA replication can be performed by physical or chemical treatment. The physical treatment can be γ radiation or exposure to UV light. The chemical treatment can be chemicals that bind to chromosomal DNA, including actinomycin D, etoposide, DNA chelating and interacting agents, and other chemotherapeutics.

In yet another embodiment, there is provided a method of forming diploid therapeutic cells by fusing non-replicating target cells with aged somatic cells to induce reprogramming therein with the steps of a. providing target cells; b. disabling DNA replication in the target cells; c. providing autologous somatic cells; d. combining the non-replicating target cells and the autologous somatic cells; e. adding to the combination polyethylene glycol to fuse the cells wherein the non-replicating target cells reprogram the genome of the somatic cells; f. growing the fused in appropriate cell medium to produce diploid cells that have the morphology and function of target cells; and g. selecting autologous reprogrammed diploid cells that have the morphology and function of target cells, thereby providing reprogrammed autologous diploid cells to the donor for use in cell replacement therapy and cosmetics. The step of disabling DNA replication can be performed by physical or chemical treatment. The physical treatment can be γ radiation or exposure to UV light. The chemical treatment can be chemicals that bind to chromosomal DNA, including actinomycin D, etoposide, DNA chelating and interacting agents, and other chemotherapeutics. The target cells can be exogenous ESCs, adult stem cells or insulin-secreting cells. The somatic cells can be mature skin cells, blood cells and bone marrow cells.

In another embodiment, there is provided a method of performing cell therapy and cosmetic applications that would otherwise use ESCs or tissue stem cells. First, ESL cells are provided; then the cell therapy or cosmetic applications are performed, but the ESCs or tissue stem cells are replaced with the patient-specific diploid ESL cells.

In another embodiment, there is provided a method of locally rejuvenating human skin with the steps of a. pretreating skin with an agent that permeabilizes the skin cells; b. applying rejuvenating agents selected from novacell extracts, ESC extracts, stem cell extracts, egg extracts, recombinant proteins or a combination thereof; c. maintaining the skin contact with the rejuvenating agent; and d. repeating steps a-c as necessary. This method can be preceded with sterilizing the skin. The rejuvenating agent can contain a cell or nuclear extract, to either of which is added albumin, ATP, phosphocreatine, creatine kinase, RNase inhibitor and nucleotide phosphates.

In yet another embodiment, there is provided a method of preparing rejuvenating factors from whole cells, with the steps of a. providing novacells, ESCs, tissue stem cells, eggs or cells obtained from embryo, fetus, fetal tissues, recombinant proteins or a combination thereof; b. isolating the cells to be extracted; c. subjecting the cells to at least two freeze-thaw cycles; d. centrifuging the cells at high speed; and e. drawing off the supernatant that comprises the rejuvenating factors.

In still another embodiment, there is a method of preparing rejuvenating factors from nuclei, with the steps of a. providing novacells, ESCs, tissue stem cells, eggs or cells obtained from embryo, fetus, fetal tissues, recombinant proteins or a combination thereof; b. isolating the cells to be extracted, comprising fetal cells and/or ESCs; c. lysing the cells in hypotonic buffer; d. centrifuging to separate off the buffer; e. subjecting the remaining nuclei to at least two freeze-thaw cycles; f. centrifuging to obtain an extract of the nuclei; and g. optionally concentrating the rejuvenating factors by dialysis.

In yet another embodiment, there is provided a method of locally rejuvenating an organ, with the steps of a. providing rejuvenating agents prepared from cells or nuclei; and b. administering the rejuvenating agents periodically into the organ, until improvement in signs, symptoms or test results, thereby slowing aging of the organ and/or improving its function. The method of administering can be by an intravenous, subcutaneous, intraperitoneal, intramuscular, intraventricular, intratracheal, intraarticular, interpericardial, intrapulmonary, intranasal or intraarterial route. Using the intranasal route, the rejuvenating agents can be placed into the nasal cavity, whereby the rejuvenating agents reach the central nervous system to treat neural degenerative diseases.

In another embodiment, there is provided a method of rejuvenating mammalian somatic cells and differentiating them into desired cells by an abbreviated process with the steps of a. treating somatic cells with a pore-opening treatment and washing with a physiological buffer; b. exposing the somatic cells of step a to rejuvenating buffer, ESC nuclear extract and desired cell extracts for about one hour; c. growing the cells of step b in KO-DMEM solution optionally supplemented with 20% FBS, penicillin, streptomycin, glutamine, non-essential amino acids, β-mercaptoethanol, bFGF, TGF-β1 and LIF; d. growing the cultured cells in inverted hanging droplets on the cover of a plate for about two hours to overnight; e. collecting and combining the inverted droplets; and f. growing the collected cells on gelatin-coated plates in DMEM supplemented with the desired cell-producing agents until the cells form the desired cell types. In one variation the somatic cell comprises fibroblasts, the desired cell type is muscle cells, and the desired cell-producing agents include 2% inactivated horse serum, filtered supernatant from myoblast cell culture medium, whereby the collected cells are so exposed until forming myotubes. In this method step a treating the fibroblasts can include trypsin-EDTA, streptolysin O, electroporation or virus-mediation.

In another embodiment, there is provided a rejuvenating buffer composition containing rejuvenating factors from cells or nuclei or a combination thereof; 1 mg/ML albumin; 1 mM ATP; 5 mM phosphocreatine; 25 μg/mL creatine kinase; 0.4 U/mL RNase inhibitor; and 1 mM each of the 4 dNTPs. Preferably the combination extract is about one half fetal cell extract and one half nuclear extract of ESCs. Preferably the cell or nucleic extract is cell free.

In one embodiment, a method of systematically rejuvenating a mammalian body and improving general health and immune function has the steps of providing rejuvenating agents from nova cell extracts, stem cell extracts, egg extracts, nova cells, ESCs, stem cells, recombinant proteins or a combination thereof; and administering the rejuvenating agents periodically, as indicated by improvement. The route of administration can be intravenous, intraperitoneal, intramuscular, intrathecal, intranasal routes or a combination thereof.

In another embodiment a method of rejuvenating cells that have undergone many passages in tissue culture has the steps of providing previously cultured cells; providing a rejuvenating extract, albumin, ATP, phosphocreatine, creatine kinase RNase inhibitor and nucleotide phosphates; combining the cells with the rejuvenating solution and incubating for a sufficient time for the constituents of the rejuvenating solution to penetrate the cells; adding a solution of cell medium, calcium chloride and optionally antibiotics; and culturing the cells to expand the cell population, thereby rejuvenating cells that have undergone many passages in tissue culture. In addition, a variation on the method replaces the last two steps with the following steps: growing the rejuvenated cells in inverted hanging droplets on the cover of a plate or an uncoated Petri dish for a sufficient time to accelerate cell aggregation, growing the cell aggregates in suspension to form EBs on 0.2% to 2% agarose or in an uncoated Petri dish, culturing the EB-like cells on feeder cells, on coated plates or disks, or on Matrigel in appropriate medium supplemented with growth factors, and selecting cell colonies with the same morphology as stem cells, whereby the previously cultured cells are dedifferentiated into pluripotent cells for further tissue culture.

A method of treating liquid cancers, leukemia, lymphomas and hematopoietic dysfunction, whether or not caused by chemotherapy regimens, has the steps of first providing rejuvenated cells prepared by providing aged somatic cells; providing a rejuvenating solution comprising rejuvenating extract, albumin, ATP, phosphocreatine, creatine kinase, RNase inhibitor and nucleotide phosphates; combining the aged somatic cells with the rejuvenating solution and incubating for a sufficient time for the constituents of the rejuvenating solution to penetrate the cells; adding a solution of cell medium, calcium chloride and optionally antibiotics to expand the cell population; separating the rejuvenated cells; and combining the rejuvenated cells with a physiological solution to prepare an administrable preparation. Next the rejuvenated cells are administered to a mammal suffering from liquid cancers, leukemia, lymphomas and hematopoietic dysfunction, whether or not caused by chemotherapy regimens.

In another embodiment, there is disclosed a method of treating a mammal with CNS trauma, stroke, Alzheimer's Disease, Parkinson's Disease, or amyotrophic lateral sclerosis. The first step is providing rejuvenated cells which are prepared by i. providing a sample of aged somatic cells; ii. providing a rejuvenating solution comprising rejuvenating extract, albumin, ATP, phosphocreatine, creatine kinase, RNase inhibitor and nucleotide phosphates; iii. combining the somatic aged cells with the rejuvenating solution and incubating for a sufficient time for the constituents of the rejuvenating solution to penetrate the cells; iv. adding a solution of cell medium, calcium chloride and optionally antibiotics to expand the cell population; v. separating the rejuvenated cells; and vi. combining the rejuvenated cells with a physiological solution to prepare an administrable preparation. The next step is administering the rejuvenated cells to a mammal suffering from CNS trauma, stroke, Alzheimer's Disease, Parkinson's Disease, or amyotrophic lateral sclerosis.

In another embodiment, there is a kit for rejuvenating aging cells, containing a. an agent for opening aging cell pores, selected from trypsin and streptolysin O; b. a rejuvenating buffer composition; and c. a cell-free extract of fetal cells and ESC nuclei. The extract can be replaced with an mRNA extract from pluripotent cells. The pluripotent cells can be ESCs, PGCs, fetal cells, eggs, fertilized eggs, umbilical cord blood stem cells, tissue stem cells, blastocyst cells, or a combination thereof.

In yet another embodiment there is provided a method of rejuvenating somatic cells, the method comprising the steps of a. providing at least one human ES cell transcription factor in a mammalian expression vector; b. transfecting the vector of step a into exponentially growing human cells; c. after transfection, isolating the cells expressing the vector; d. growing the isolated vector-expressing cells on plates until confluence; e. collecting the vector-expressing cells; f. treating the vector-expressing cells with membrane-permeabilizing solution and ES cell extracts; g. sealing the membranes of extract-treated cells; and placing the extract-treated cells in hanging droplets to grow and isolating fully reprogrammed cells in clusters. The human ES cell transcription factors are Oct4, Sox2 and Nanog.

### BRIEF DESCRIPTION OF DRAWINGS

FIG. 1 summarizes the inventive method of obtaining mature cells from individuals, culturing the cells, rejuvenating the cells into “novacells” and subsequently using in cell replacement therapy.

FIGS. 2A-2F show control fibroblasts (A, B and C) and novacells rejuvenated with fetal extract (D), novacells rejuvenated with ESC nuclear extract (E) and rejuvenated fibroblasts in inverted droplets (F). FIGS. 2G-2I show control aged bone marrow stromal cells (G) and novacells rejuvenated with fetal extract (H) and nova cells rejuvenated with ESC nuclear extract (I).

FIGS. 3A and 3B are photomicrographs of an untreated skin biopsy (A) and rejuvenated skin biopsy (B) with more cell proliferation.

FIG. 4A shows unrejuvenated FNSK2 cells; FIGS. 4B-4F show, in different stages, novacells formed from FNSK2 cells. 4B shows early FN-ESL novacells; 4C shows FN-ESL novacells in an embryoid body; 4D shows further growth on a matrigel-coated plate; 4E shows further growth on agarose gel; and 4F shows the FN-ESL novacells on a layer of feeder cells.

FIG. 5 is a photograph of a gel, indicating that the FNSK2 fibroblasts do not produce the three embryonic stem cell-specific biomarkers (Oct4, Ndp52L1 and DPPA3) and that the three stages of rejuvenated cells all produce those biomarkers.

FIGS. 6A-6I show the starting mature fibroblasts and novacells resulting from a variety of protocols. FIGS. 6B-6E show the novacells resulting from various methods of treating the mature cells to make them permeable to rejuvenating factors. FIGS. 6F-6K show the novacells resulting from a variety of rejuvenating extracts.

FIGS. 7A-7D show fusion novacells produced from fibroblasts from a mature male and replication-defect stem cells whose replication was impaired by radiation or actinomycin D.

FIG. 8 is a summary of the rejuvenation and differentiation process for producing novacells for cell therapy.

FIGS. 9A-9G show differentiation of novacells into neural precursors, neural cells, insulin-secreting islands, C-peptide positive islands, beating cardiomyocytes, skeletal myocytes and adipocytes, respectively.

FIGS. 10A-10D show WTCL unrejuvenated tumor cells, novacells on feeder cells, ESL novacell colonies and a ESL novacell colony on feeder cells, respectively. After rejuvenation, tumor cells show less or no tumor-producing capacity as shown by fewer agar-gel-forming colonies and no tumors in nude mice. This rejuvenation-induced dedifferentiation may provide a breakthrough strategy to develop tumor therapies.

FIGS. 11A-11C show the results of a one-step rejuvenation/differentiation protocol turning mature fibroblasts into rejuvenated myocytes.

FIG. 12 is gel blot illustrating reactivation of telomerase in cells that underwent the rejuvenation process; lanes 2, 3 and 7 show untreated controls; and lanes 4 and 5 show the results of rejuvenation and compare favorably with the positive control of ESCs in lane 6.

FIG. 13 summarizes the procedure of using novacells to produce in vivo rejuvenation.

FIGS. 14A and 14B show untreated skin scars and faded scars after in vivo rejuvenation.

FIGS. 15A-14D show mice that include control aged mice (A and C). FIGS. 15B and D show the more active rejuvenated mice.

FIG. 16 is a schematic of the preprogramming pES vector. Restriction enzymes were used to clone the transcription factors into the pEGFP-N3 vector.

FIGS. 17A, 17B, and 17C show fibroblasts, pluripotent embryonic stem-like (ESL) cells by a tow-step in vitro epigenetic reprogramming growing on feeder cells, and ESL cells growing on a Matri gel-coated plate, respectively.

### DETAILED DESCRIPTION

The present invention describes methods of rejuvenating aged cells into “novacells”, which are much younger and more potent than the original cells. Novacells become totipotent, pluripotent or multipotent. The rejuvenated cells have restored function lost during aging and thus are useful in cell replacement therapies of human diseases. When the methodology is applied in vivo, cell rejuvenation will slow or stop the aging process of tissues, organs and the whole body.

A major advantage of this invention is that it rejuvenates cells or tissues from the patient who will receive the rejuvenated cells. With such autologous cells and tissues, there is no risk of developing graft-versus-host rejection. Cells to be rejuvenated may be collected from a variety of sources, including skin, blood or bone marrow.

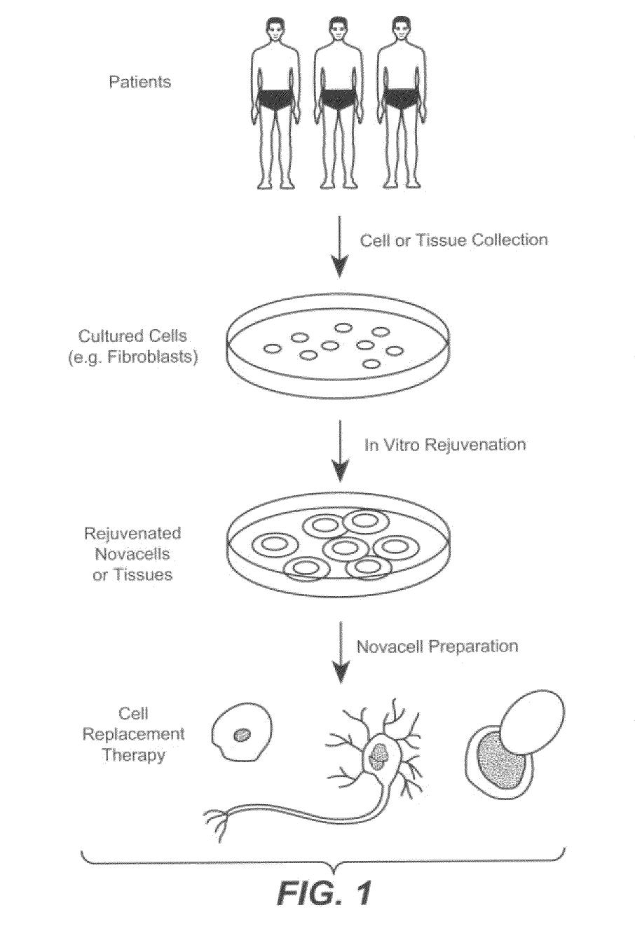

FIG. 1 is a schematic outline of the procedure to rejuvenate aged cells into potent novacells in vitro. Cells are first collected from an elderly person (e.g., from the skin, blood, bone marrow or biopsy tissues) and are cultured in appropriate media to expand the cell population. Cells are optionally exposed to a cell membrane permeabilizing reagent (e.g., trypsin/EDTA) to open the gap junction of the cells. After centrifugation and separation of the permeabilizing reagent, the cells are rejuvenated with the rejuvenating factors in the rejuvenating buffer. After incubation at 37° C. for a short time (about 30 minutes to 3 hours), cells are grown in the medium in the presence of fetal bovine serum (FBS) and antibiotics. The rejuvenated cells have enhanced physiological function and grow at a faster rate than the starting aged cells. These rejuvenated novacells are useful in cell therapy, including cosmetic applications to the skin.

Bone marrow stromal cells, the non-hematopoietic cells of mesenchymal origin that support hematopoiesis, are multipotential and self-replicating in culture. Like ESCs, these progenitors can be differentiated into many other cell types, like osteoblasts, chondroblasts, adipocytes, cardiomyocytes, neuron-like cells and astrocytes. The plasticity of stromal cells is the basis for potential use in cell replacement therapy.

However, aging also is an important determinant of the growth of bone marrow stromal cells in cell culture. The stromal cells isolated from aged mice grow more slowly than those isolated from young mice. It is thus desirable to rejuvenate those bone marrow stromal cells in vitro before they are used in cell replacement therapy. It may also be desirable to rejuvenate bone marrow cells to enhance the growth and recovery of bone marrow prior to transplantation in the treatment of leukemia and other hematopoietic diseases.

Stem cells are defined as pluripotential cells that have the ability to self-renew and to differentiate to mature cells of a particular tissue (Morrison et al, Ann Rev Cell Dev Biol 1995, 11:35-71). One of the features of ESCs is their ability to differentiate into other cells in differentiating media. ESCs also grow into undifferentiated EBs.

In general the method for rejuvenating aged cells into potent novacells has the steps of rejuvenating the cells into potent novacells in vitro with rejuvenating reagents containing tissue and cell components derived from cells in early stages of development, e.g., embryos, fetuses, blastocysts, ESCs, stem cells, cord blood stem cells and eggs. The source of the rejuvenating extract is generally younger than the cell to be rejuvenated.

The resulting novacells are younger-acting and more proliferative than the original cells in morphology, physiology and functionality. These novacells have enhanced the function over the starting cells in vitro and in vivo. Examples of these youthful actions include but are not limited to making more collagen and elastin and more proliferative red blood cell precursors and bone marrow cells. Preferably the novacells have the features of ESCs in morphology, physiology, functionality and pluripotency. These novacells can be used to replace ESCs in research and commercial applications, e.g., treating specific diseases, creating new compatible organs and tissues and screening new therapeutic drugs.

A wide variety of somatic cells can be used in the methods taught here, including but not limited to fibroblasts, lymphocytes, epithelial cells, endothelial cells, skeletal, cardiac and smooth muscle cells, hepatocytes, pancreatic islet cells, bone marrow cells, astrocytes, and non-embryonic stem cells (i.e., tissue stem cells). The procedure also is useful to rejuvenate cells that have undergone many passages in tissue culture.

The novacells can be used to replace ESCs to differentiate into the desired tissues or tissue-specific precursor cells in cell therapy. The rejuvenated novacells are also useful for transplantation into a specific organ or tissue of human or animal to treat disease.

The term rejuvenating agent refers to factors that are able to reprogram cells and rejuvenate them into cells of early stages of development, e.g., newborn, fetal, embryo, and ESCs. Rejuvenating agents used in the invention include but are not limited to tissue extracts, nuclear extracts or cell extracts containing rejuvenating factors being capable of rejuvenating somatic cells into pluripotent novacells. The rejuvenating agent contains tissue extracts of embryos, newborns, newborn tissues, fetuses, placentas, and fetal liver and other tissues. The nuclear extracts can be obtained from ESCs, stem cells, cord blood stem cells, germ cells and primordial germ cells (PGCs), eggs, fertilized eggs, embryos, newborn tissues, fetal liver, other fetal tissues and other immature tissues. Rejuvenating factors can also be recombinant proteins and recombinant cDNA and DNA alone or in vectors (e.g., plasmid or viral). In another aspect of the present invention, the rejuvenating agent contains mRNA or total RNA derived from ESCs, stem cells, cord blood stem cells, PGCs, eggs, fertilized eggs, embryos, newborn tissues, fetal liver, and other tissues. Introducing these mRNA or total RNA into somatic cells permits the mRNA to synthesize factors inside the cells where they epigenetically reprogram the genome and rejuvenate the cells into totipotent or pluripotent cells. Alternately, the rejuvenating agent contains newborn serum and recombinant proteins cloned from ESCs, PGCs, eggs, fertilized eggs, embryos, newborn tissues, newborn serum, placenta, fetal liver and other fetal tissues.

Novacells are defined as rejuvenated cells that act much younger and are more proliferative than cells that have not been rejuvenated. Furthermore, novacells demonstrate improved functionality and have an extended lifespan. Novacells have enhanced telomerase activity and thus should longer telomeres. These cells may live for unlimited passages without early senescence.

The novacells synthesize more biological compounds, including but not limited to proteins, enzymes, hormones, and growth factors. Thus, novacells are useful in restoring the functions of specific cells, tissues, and organs. For example, the aged skin fibroblasts fail to produce or actually make less collagens and elastins, causing skin wrinkles. The rejuvenated fibroblasts function like those of fetal and newborn skin cells and produce more collagens and elastins when implanted or injected into skins to treat wrinkles in older people. The rejuvenated blood cells, such as bone marrow cells and hematopoietic cells, are more proliferative and longer lived, and thus are beneficial in aplastic anemia, congenital anemia, chemotherapy-caused anemia and other blood disorders.

A variety of administration methods can be used, depending on the therapeutic objective. The methods of delivery may vary but include and are not limited to intravenous, subcutaneous, intraperitoneal, intramuscular, intraspinal, intra-cerebroventricular, intra-tracheal and intra-articular (into joints). The rejuvenating factors also can applied to the skin or placed in a skin patch, particularly one that increases skin permeability.

The term “effective amount” is used herein to describe concentrations or amounts of components such as differentiation agents, precursor or progenitor cells, specialized cells, such as neural cells, and/or other agents which are effective for producing an intended result including differentiating stem and/or progenitor cells into specialized cells, such as neural, or other cell types. Compositions according to the present invention may be used to effect a transplantation of the novacells within the composition to produce a favorable change in the brain or spinal cord, or in the disease or condition being treated, whether that change is stabilization or an improvement (e.g., stopping or reversing various degenerative diseases or conditions, including a neurological deficit.

The term “administration” or “administering” is used throughout the specification to describe the process by which cells of the subject invention, such as novacells, or differentiated cells obtained therefrom, are delivered to a patient for therapeutic purposes. Cells of the subject invention are administered via a variety of routes including, but not limited to, parenteral, intrathecal, intraventricular, intraparenchymal (including into the spinal cord, brainstem or motor cortex), intracisternal, intracranial, intrastriatal, oral, topical and intranigral routes, among others. Basically any method can be used so that it allows cells of the subject invention to reach the ultimate target site. Cells of the subject invention can be administered in the form of novacells or differentiated cells. The compositions, according to the present invention, may be used without treatment with a differentiating agent (“untreated” i.e., without further treatment in order to promote differentiation of cells within the novacell sample) or after treatment (“treated”) with a differentiation agent or other agent which causes certain stem and/or progenitor cells within the novacell sample to differentiate into cells exhibiting a differentiated phenotype, such as a neuronal phenotype. The cells may undergo ex vivo differentiation prior to administration into a patient.

Administration often depends upon the disease or condition treated and may preferably be via a parenteral route, e.g., intravenously, by administration into the cerebrospinal fluid, by nasal inhalation, by direct implantation into the affected tissue, or by other systemic or topical means. For example, in the case of Alzheimer's disease, Huntington's disease, and Parkinson's disease, the preferred route of administration will be a transplant directly into the CNS (e.g., the striatum, the substantia nigra or both for Parkinson's disease). In the case of amyotrophic lateral sclerosis (Lou Gehrig's disease) and multiple sclerosis, the anticipated preferred route of administration is injection into the cerebrospinal fluid.

The terms “grafting” and “transplanting” and “graft” and “transplantation” are used throughout the specification synonymously to describe the process by which cells of the subject invention are delivered to the site where the cells are intended to exhibit a favorable effect, such as repairing damage to a patient's central nervous system (which can reduce a cognitive or behavioral deficit caused by said damage), treating an acute or subacute neurodegenerative disease, nerve damage caused by cerebrovascular accident (stroke) or physical injury (trauma). Cells of the subject invention can also be delivered in a remote area of the body by any mode of administration known to those experienced in the art, relying on cellular migration to the appropriate area(s) to effect transplantation.

### Molecular Biology Techniques

Standard molecular biology techniques known in the art and not specifically described are generally followed as in Sambrook et al., MOLECULAR CLONING: A LABORATORY MANUAL, Cold Springs Harbor Laboratory, NY (1989, 1992), and in Ausubel et al., CURRENT PROTOCOLS IN MOLECULAR BIOLOGY, John Wiley and Sons, Baltimore, Md. (1989). Polymerase chain reaction (PCR) methodology is generally employed as specified as in Jam et al, PCR PROTOCOLS: A GUIDE TO METHODS AND APPLICATIONS, Academic Press, San Diego, Calif. (1999). Reactions and manipulations involving other nucleic acid techniques, unless stated otherwise, are performed as generally described in Sambrook et al, MOLECULAR CLONING: A LABORATORY MANUAL, Cold Springs Harbor Laboratory Press, and methodology as set forth in U.S. Pat. Nos. 4,666,828; 4,683,202; 4,801,531; 5,192,659; and 5,272,057, and incorporated herein by reference. In situ PCR in combination with flow cytometry can be used for detection of cells containing specific DNA and mRNA sequences (e.g., Testoni et al, 1996, Blood, 87:3822).

Standard methods in immunology known in the art and not specifically described herein are generally followed as set forth in Stites et al (Eds.), BASIC AND CLINICAL IMMUNOLOGY, 8 Ed., Appleton & Lange, Norwalk, Conn. (1994); and Mishell and Shigi (Eds.), SELECTED METHODS IN CELLULAR IMMUNOLOGY, W.H. Freeman and Co., New York (1980).

### Immunoassays

In general, immunoassays are employed to assess a specimen for cell surface markers or the like. Immunocytochemical assays are well known to those skilled in the art. Both polyclonal and monoclonal antibodies can be used in the assays. Where appropriate other immunoassays, such as enzyme-linked immunosorbent assays (ELISAs) and radioimmunoassays (RIA), are well known to those skilled in the art and can be used. Available immunoassays are extensively described in the patent and scientific literature. See, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771; and 5,281,521 as well as Sambrook et al, MOLECULAR CLONING: A LABORATORY MANUAL, Cold Springs Harbor, N.Y., 1989. Numerous other published, scientific references are readily available to those skilled in the art.

### Gene Therapy

Gene therapy as used herein refers to the transfer of genetic material (e.g., DNA or RNA) of interest into a host to treat, prevent, or modify any number of diseases or conditions. The genetic material of interest encodes a product (e.g., a protein, polypeptide, peptide, functional RNA, and/or antisense molecule) whose in vivo production is desired. For example, the genetic material of interest encodes a hormone, receptor, enzyme polypeptide or peptide of therapeutic value. Alternatively, the genetic material of interest encodes a suicide gene. For a detailed review see “Gene Therapy” in ADVANCES IN PHARMACOLOGY, Academic Press, San Diego, Calif., 1997.

### Administration of Cells for Transplantation

The novacells of the present invention can be administered and dosed in accordance with good medical practice, taking into account the clinical condition of the individual patient, the site and method of administration, the scheduling of administration, the patient's age, sex, body weight and other factors significant to medical practitioners. The pharmaceutically “effective amount” or dosage schedule for purposes herein is to be determined by such considerations as are known to those skilled in experimental clinical research, pharmacological and clinical medical arts. The amount must be effective to achieve stabilization, improvement (including but not limited to youthful appearance and function) or elimination of symptoms and other indicators as are selected as appropriate measures of disease progress, regression or improvement by those skilled in the art.

In the method of the present invention, the novacells can be administered by various routes as would be appropriate to implant the novacells in the CNS or other tissues or organs. Routes include, but are not limited to, parenteral administration, including intravenous and intraarterial administration, intrathecal administration, intraventricular administration, intraparenchymal, intracranial, intracisternal, intrastriatal, and intranigral administration, as well as oral and topical administration.

Pharmaceutical compositions comprising effective amounts of novacells are also contemplated by the present invention. These compositions comprise an effective number of cells, optionally, in combination with a pharmaceutically acceptable carrier, additive or excipient, and are suspended in one or more appropriate media. In certain aspects of the present invention, to the patient in need of a transplant, cells are administered in sterile saline. In other aspects of the present invention, the cells are administered in Hanks Balanced Salt Solution (HBSS), Isolyte S, pH 7.4 or other such fluids chosen from 5% dextrose solution, 0.9% sodium chloride, or a mixture of 5% dextrose and 0.9% sodium chloride. Other examples of diluents are chosen from lactated Ringer's solution, lactated Ringer's plus 5% dextrose solution, Normosol-M and 5% dextrose, and acylated Ringer's solution. Still other approaches may also be used, including the use of serum-free cellular media. Systemic administration of the cells to the patient may be preferred in certain indications; whereas, direct administration at the site of or in proximity to the diseased and/or damaged tissue may be preferred in other indications, as determined by the pharmaceutical presentation and as determined by those skilled in the art. Also contemplated is the administration of the subject cells by various additional media, including injectable or implantable pellets, etc.

Pharmaceutical compositions according to the present invention preferably comprise an effective number of novacells within the range of about 1.0×10⁴ cells to about 1.0×10¹⁴ cells, more preferably about 1×10⁵ to about 1×10¹³ cells, even more preferably about 2×10⁵ to about 8×10¹² cells. Said cells are generally administered in suspension, optionally in combination with a pharmaceutically acceptable carrier, additives, adjuncts or excipients, as required to achieve a pharmaceutically acceptable result.

Throughout this application, various patents and patent publications are referenced. The disclosures of all of these patents and patent publications cited within those publications in their entireties are hereby incorporated by reference into this application in order to more fully describe the state of the art to which this invention pertains. The following examples are not intended to limit the scope of the claims of the invention, but are rather intended to be exemplary of certain embodiments. Any variations in the exemplified methods which occur to the skilled artisan are intended to fall within the scope of the present invention.

### EXAMPLES

The schematic outline of the procedure to rejuvenate aged cells into potent novacells in vitro is shown in FIG. 1. First, the cells are collected from a mature person, e.g., from skin, blood, bone marrow or biopsy tissue, and are cultured in appropriate medium to expand the cell population. Next, cells are exposed to a cell membrane-permeabilizing reagent, e.g., trypsin/EDTA, to open the gap junction of the cells. After centrifugation, cells are rejuvenated with the rejuvenating factors in the rejuvenating buffer. While not wishing to be bound by any theory, the rejuvenating factors are believed to enter the nuclei and remodel chromatin. Epigenetic reprogramming (e.g., DNA methylation and histone modifications) activates genes related to cell growth and aging. After incubation with these factors, cells are grown in medium in the presence of fetal bovine serum (FBS) and antibiotics. The rejuvenated cells have enhanced physiological functionality (such as telomerase or telomere length, growth factor expression, collagen synthesis and cell replication capacity) and grow at a faster speed as compared with the original aged cells. These rejuvenated novacells are useful in cell therapy including cosmetic applications. Cell therapies vary with the novacells differentiated in vitro. Examples of uses include but are not limited to liver failure, peptic ulcers, burns, leukemia and chemotherapy-related anemia.

### Example 1

### Culturing Skin Fibroblasts

After sterilization, a skin biopsy (2 mm²) was cut from the inner forearm of a male volunteer aged 49 years. The skin biopsy was cut into several small pieces with a sterilized razor and directly placed into a 6-well plate, where it was covered with a thin layer of DMEM medium (Invitrogen, Carlsbad, Calif.), supplemented with 10% fetal bovine serum (FBS) and 100 U/mL of penicillin and 100 μg/mL of streptomycin, and grown at 37° C. in room air supplemented with 5% CO₂. The medium was replaced with fresh DMEM daily.

After approximately 2 wk of incubation, fibroblasts had begun to grow around the skin edges. Fibroblasts were detached with 1× trypsin-EDTA (Invitrogen). The trypsin/fibroblast solution was centrifuged at 1200 rpm for 3 min. The fibroblast pellet was resuspended and the cells were counted. Depending on the count, the cells were seeded in a new 6- or 24-well plate in DMEM medium. The fibroblasts were collected and transferred to 75 mm plates or flasks for further expansion. These aged fibroblasts grew more slowly and made less collagen and elastin protein than rejuvenated cells (see below). Cells were again trypsinized, centrifuged and resuspended in 10% FBS and 8% DMSO. This cell solution was stored in liquid nitrogen.

### Example 2

### Culturing Blood or Bone Marrow Cells

The success in bone marrow transplantations declines with age, so one can infer that younger (neonatal) cells are preferable for hematopoietic reconstitution. Similarly, aging is also an important determinant of the growth of bone marrow stromal cells in cell culture. The stromal cells isolated from aged mice grow much more slowly than those isolated from young mice. It is thus desirable to rejuvenate aged bone marrow cells in vitro before they are used in cell replacement therapy.

White blood cells provide a quick and convenient source of terminally differentiated cells that can be used for in vitro rejuvenation. A 10 mL blood sample was collected using sodium heparin as the anticoagulant and was added to a 15 mL tube and diluted in four volumes of phosphate-buffered saline (PBS) containing EDTA (3 mM). The diluted blood was loaded onto Ficoll-Hypaque medium (Sigma, St. Louis Mo.) in a 50-mL conical tube and centrifuged at 400 rpm for 30 min at 20° C. in a swinging-bucket rotor without break. The upper layer (plasma) was removed and the interphase cell layer (containing lymphocytes and monocytes) was carefully removed to a second 50 mL tube. PBS containing 2 mM EDTA was added to a total volume of 30 mL and was centrifuged at 300 rpm for another 10-20 min. This wash step was repeated, and the cell pellet was resuspended in 300 μL degassed buffer (PBS, pH 7.2, supplemented with 0.5% bovine serum albumin [BSA] and 2 mM EDTA). The cells were suspended in DMEM medium (Invitrogen) on 75-100 mm plates. The live mononuclear cells, including stem cells attached to the plate in about 30 min. The remaining red blood cells and other white blood cells were still suspended in the medium and were washed away by a simple change of the medium. Cells attached to the plate were trypsinized and used for rejuvenation. Optionally, CD34-positive progenitor cells can be further isolated using the MiniMacs isolation kit (Miltenyi Biotec, Auburn, Calif.). The white cell pellet was collected and cultured in Myelocult medium (H1500, Stem Cell Technologies, Vancouver, BC, Canada) supplemented with 10% FBS and human cytokines including stem cell factor (SCF, 10 ng/mL), Flt3 ligand (FL, 10 ng/mL), interleukin-3 (IL-3, 20 ng/mL), IL-6 (10 ng/mL), IL-11 (10 ng/mL), thrombopoietin (TPO, 50 ng/mL) and erythropoietin (EPO, 4 units/mL). The cytokines were purchased from EMD Biosciences, San Diego, Calif., and BD BioSciences, San Jose, Calif. The culture was incubated at 37° C. in air supplemented with 5% CO₂.

### Example 3

### Preparation of Fetal Extracts as the Rejuvenating Factor

Tissues collected in the early stages of development (e.g., fetus and embryo) are excellent sources of rejuvenating factors for rejuvenating cells. The following example of mouse fetal liver illustrates the procedure.

A fetus was collected from a pregnant mouse and the fetal liver was dissected into a Petri dish containing ice-cold PBS. The liver tissue was minced with sterile scissors or razors into small pieces, which were transferred with PBS into a glass homogenizer. The liver tissue was homogenized as the pestle gently moved up and down about 20 times. The cells were passed through a nylon layer to remove fibrous connective tissues and were centrifuged at 600 rpm at 4° C. for 10 min. Cells were washed twice with ice-cold extraction buffer (50 mM HEPES, pH 7.4, 50 mM KCl, 5 mM MgCl₂, 2 mM (3-mercaptoethanol, and 5 mM EGTA). The cells were washed with the same buffer containing additionally the following protease inhibitors: cytochalasin B, leupeptin, aprotinin, and pepstatin A (10 μg/mL each). After 5 min of incubation on ice, cells were centrifuged at 1000 rpm for 1 min. The supernatant was carefully removed to leave approximately one half the solution volume.

Cells were put through 3 freeze/thaw cycles (−80° C. to room temperature) and were centrifuged at 12,000 rpm at 4° C. for 30 min. The supernatant extract was collected and 2% glycerol was added. Aliquots (0.1 mL) in 0.6 mL tubes were frozen in liquid nitrogen and stored at −80° C.

Fetal and embryonic extracts, such as this, are very rich in factors for rejuvenating aged cells, tissues, organs and the whole mammalian body. This method also can be used by extracting other fetal tissues, the whole fetus, embryos and placenta.

### Example 4

### Preparation of Embryonic Stem Cells (Escs) for Rejuvenating Factors

ESCs were trypsinized (see Example 1), and approximately 2×10⁷ cells were collected in a 1.5 mL tube. As described in Example 3, cells were first washed twice with ice-cold extraction buffer and were then subjected to 3 freeze/thaw cycles (−80° C. to room temperature), and were centrifuged at 12,000 rpm at 4° C. for 30 min. The supernatant extract was collected and 2% glycerol was added. Supernatant aliquots (0.1 mL) in 0.6 mL tubes were frozen in liquid nitrogen and stored at −80° C.

This method can also be used to isolate cell extracts from other tissue stem cells, cord blood stem cells and rejuvenated new cells for use in cell rejuvenation. This method will also be used for extracting tissue extracts from tissues, like fetus, embryos and fetal tissues.

### Example 5

### Preparation of Rejuvenating Factors from Esc Nuclear Extracts

Nuclear extracts of ESCs were purified using the method as previously described (Tian et al, DNA Repair (Amst), 2002, 1:1039-49). Briefly, ESCs were harvested by centrifugation at room temperature at 2200 rpm for 5 min and washed once with 5 times the cell volume of cold PBS. ESCs were suspended in 5× cell volumes of Buffer A, a hypotonic buffer of 10 mM HEPES buffer, 1.5 mM MgCl₂, 10 mM KCl, 0.5 mM DTT. Maintained at 4° C., the ESCs were lysed using a glass homogenizer (Wheaton A Dounce homogenizer, ˜10 strokes), and the nuclei were collected by centrifugation at 2200 rpm for 15 min. Nuclear proteins were extracted by placing the nuclei in ½ nuclear volume of Buffer C (10 mM HEPES buffer, 25% glycerol, 1.5 mM MgCl₂, 420 mM NaCl, 0.5 PMSF, 0.5 mM dithiothreitol [DTT]), stirred for 30 min at 4° C. (again homogenized in the Dounce if necessary), and were put through 3 freeze/thaw cycles (−80° C. to room temperature). The suspension was centrifuged at high speed (12,000 rpm, SS-34) for 30 min at 4° C. Next the supernatant was dialyzed against >50 volumes of Buffer D (20 mM HEPES, pH 7.9, 20% glycerol, 0.2 mM EDTA, 100 mM KCl, 0.5 mM phenylmethylsulphonylfluoride [PMSF] and 0.5 DTT) in cold room. Buffer was changed and was dialyzed against >50 volumes of solution D for ˜2.5 hr. Supernatant was transferred to 30 mL Corex tubes and was spun at 10,000 rpm for 20-30 min in HB-4 rotor at 4° C. Protein concentrations were measured and adjusted to 25-30 mg/mL protein and were frozen in 0.1 mL aliquots in liquid nitrogen and stored at −80° C. for later cell rejuvenation.

This method can also be used to isolate nuclear extracts from other tissue stem cells, cord blood stem cells and rejuvenated cells.

### Example 6

### Method of Rejuvenating Aged Cells

Aged cells can be rejuvenated in vitro with cell or nuclear extracts collected from a variety of tissues or cells of a younger mammalian subject. After rejuvenation, the cells are more potent. After rejuvenation, the functionality of cells lost in the aging process was restored. Skin fibroblasts were used to exemplify the procedure.

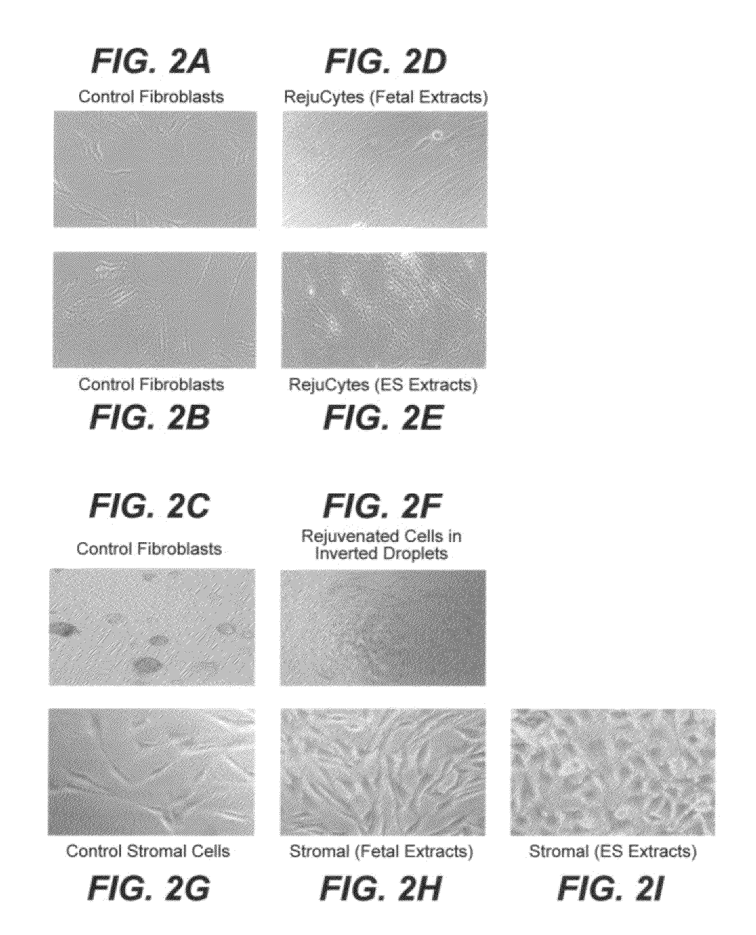

Fibroblasts were prepared as mentioned in Example 1. Briefly, fibroblasts were trypsinized and collected in aliquots of approximately 3×10⁵ cells in 1.5 mL tubes. Cells were washed with ice-cold Hank's Balanced Salt Solution (HBSS) and were pretreated with 300-1000 ng/mL streptolysin O (SLO, Sigma) at 37° C. for 1 hr to open cell gap junctions. After a wash with 200 μL ice-cold HBSS, the cells were centrifuged at 1200 rpm for 3 min at 4° C., and the cells were washed with Buffer T (20 mM HEPES, pH 7.3, 110 mM KAc, 5 mM NaAc, 2 mM MgAc, 1 mM EGTA, 2 mM DTT, 1 μg/mL each of aprotinin, pepstatin A and leupeptin. After centrifuging, the cell pellet was suspended in 20 μL of rejuvenating buffer containing 1 mg/mL BSA, 1 mM ATP, 5 mM phosphocreatine, 25 μg/ml creatine kinase (Sigma), 0.4 U/mL RNase inhibitor (Invitrogen), and 1 mM each of the four dNTPs (nucleotides triphosphate), and ESC nuclear extracts prepared in Buffer T (Martys J L et al, 1995 J. Biol. Chem. 270:25976-84; Hansis C et al, 2004 Curr Biol 14:1475-80). The cells were incubated for about 1 hr at 37° C. in a water bath with occasional tapping. After this rejuvenation step, cells were resealed by adding KO-DMEM containing 2 mM CaCl₂ and antibiotics in 6-well plates to reseal membrane pores opened by SLO. The KO-DMEM medium was changed daily until the rejuvenated fibroblasts were confluent. The cells were trypsinized and split into 100 mm plates. FIG. 2 shows control fibroblasts (FIGS. 2A and 2B) compared to novacells treated with fetal extracts (FIG. 2D) and novacells treated with ES cell nuclear extracts (FIG. 2E). The control fibroblasts had grown at a very slow rate and did not reach confluency; the cells were widely separated and sparsely distributed in the plate. In contrast, the novacells grew quickly and did reach confluency; the novacells were very crowded and lined up together. These rejuvenated cells were stored in liquid nitrogen for future usage.

Some variations are contemplated. Protein enzymes (e.g., trypsin and collagenase), detergents (digitonin) and electroporation can also be used to pretreat the cell membrane before cell rejuvenation. In a separate study (data not shown), use of one half the volume of fetal tissue extracts and one half the volume of nuclear extracts of ESCs were found to be optimal. The fetal tissue extracts appeared to function as the initiator to rejuvenate the aged cells, and the nuclear extracts of ESC worked as a booster to accelerate the rejuvenating process.

The cells rejuvenating by this procedure kept the same morphology as the original untreated cells. However, the rejuvenated cells had a higher cell proliferation rate and better cell function than control cells. For example, the rejuvenated fibroblasts synthesized more collagens and elastins than untreated fibroblasts, indicating value in cosmetic therapies (see below for data). The rejuvenated bone marrow cells or hematopoietic stem cells will be useful in the treatment of liquid cancers, leukemia and hematopoietic dysfunction caused by chemotherapy regimens. We also expect the rejuvenated cells will have longer telomeres and higher telomerase activity.

### Example 7

### Rejuvenation of Skin Tissue

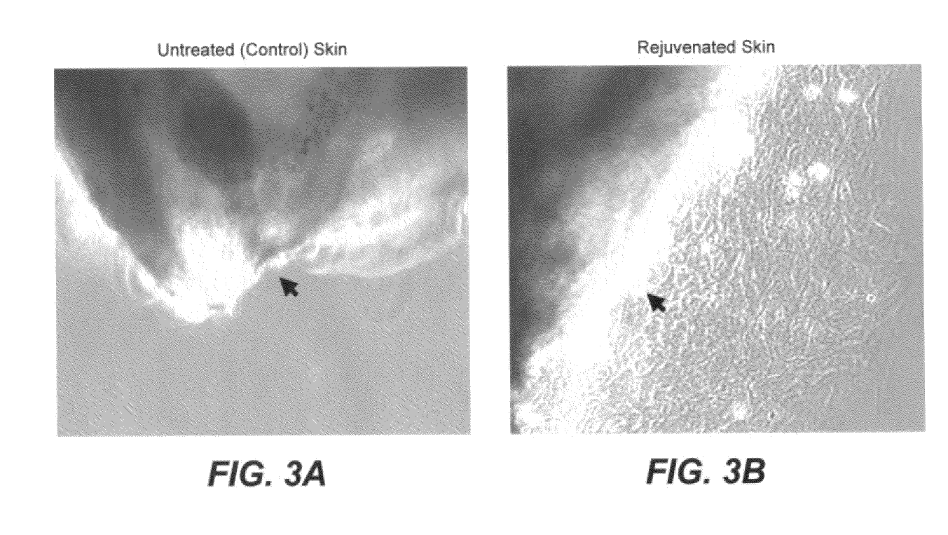

This is a shorter method of rejuvenation of aged skin tissue. A skin biopsy was obtained as in Example 1 and was grown in DMEM medium supplemented with 10% FBS and 100 U/mL of penicillin and 100 μg/mL of streptomycin. After overnight incubation, the skin tissue had stuck to the plate. The medium was removed and 50 μl, of rejuvenating buffer and 50 μL of rejuvenating factors (ESC nuclear extracts) were added to the skin tissue. Skin tissues were rejuvenated at 37° C. for 4 hr; then one volume of 2×DMEM with FBS was added. Skin tissues were incubated for 2 wk with medium replacement every 2 days. FIG. 3 shows the untreated skin on the left, with minor outgrowth of fibroblasts. Rejuvenated skin on the right has many newly growing cells emerging from the skin and attaching to the skin edge. These data demonstrate that it is possible to rejuvenate skin tissue, not just individual cells. After rejuvenation, the skin gained the function of young skin and grew more new cells. Thus, this rejuvenating procedure can be used to repair tissues or organs and restore the function of aged tissues and organs.

The above procedure was used to rejuvenate bone marrow stromal cells isolated from an aged mouse. Control bone marrow stroman cells isolated from the aged mouse grew very slowly (FIG. 2A). After rejuvenation with fetal liver extracts, stromal cells appeared very healthy and doubled at a more rapid rate (FIG. 2H). Interestingly, stromal cells rejuvenated with ESC nuclear extracts grew even better in culture (FIG. 2I).

### Example 8

### Rejuvenation of Mouse Skin Fibroblasts into Embryonic Stem-Like (ESL) Novacells

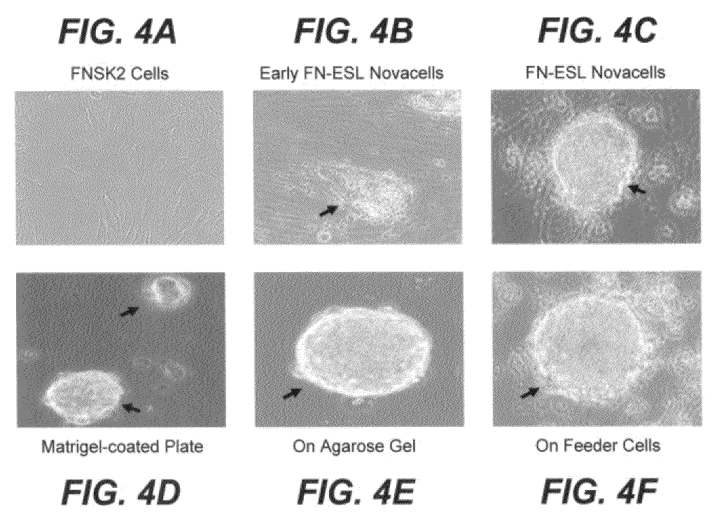

A mouse fibroblast cell line (FNSK1; Hu et al, Mol Endocrinol 1995, 9:628-36; Hu et al, J Biol Chem 1996, 271:18253-62)) was rejuvenated in vitro by the nuclear extracts of the mouse ESC (Example 5). Three selection steps were taken to dedifferentiate fibroblasts into ESL novacells. After this special selection procedure, only those fully reprogrammed cells grew in the selection medium. 1) The rejuvenated cells were first grown in inverted droplets and then in suspension on 0.35% agarose gel in Knock-Out DMEM (KO-DMEM) (Invitrogen) supplemented with 20% FBS, 1× antibiotics (100 U/mL of penicillin and 100 μg/mL of streptomycin), 1 mM glutamine, 1% non-essential amino acids, 0.1 mM 13-mercaptoethanol, 4 ng/mL bFGF, 0.12 ng/mL TGF-β1 and 10 ng/mL LIF. 2) The ESL colonies were selected and then grown on embryonic fibroblast feeder cells. After culturing, some cells became aggregated and formed small novacell colonies that have the same morphology as ESC. After growing 2 more d, the novacell colonies grew larger (FIG. 4D). 3) The cell colonies were carefully picked up and seeded on fresh feeder cells (FIG. 4F). These derived cells are called FN-ESL and were expanded and stored in liquid nitrogen, except for an aliquot saved and analyzed for ESC markers. This experiment shows that the in vitro rejuvenation method followed by the dedifferentiation protocol was able to induce dedifferentiation of fibroblasts into ESL cells. Based on their morphology and the presence of telomerase (in common with ESCs)(see Examples 9 and 20), these FN-ESL cells are expected to function like ESCs in cell therapy.

To test this hypothesis, EBs were grown from the cultured FN-ESL cells. FN-ESL cells were cultured in KO-DMEM medium with 20% FBS at 37° C. in the presence of 1000 U/mL of LIF. The exponentially growing FN-ESL were collected by trypsin detachment and the cell suspensions were made into hanging droplets (20 μL) onto the cover of an inverted Petri dish. The bottom of the dish was filled with PBS or water. After 3 d, EBs had formed and were collected onto a fresh culture dish pre-coated with 0.1% gelatin. The formation of EBs by FN-ESL shows that FN-ESL function like ESCs.

### Example 9

### Expression of ESC Markers in FN-ESL Cells

FN-ESL cells were collected from plates using trypsin-EDTA. Total RNA was extracted from cells by Tri-Reagent method (Sigma, St. Louis, Mo.). To eliminate DNA contamination in cDNA synthesis, RNA samples were first treated with DNase I; then cDNA was synthesized with RNA reverse transcriptase (Vu and Hoffman, J Biol Chem 1996, 271:9014-9023; Hu et al, Mol Endocrinol 1995, 9:628-36; Hu et al, J Biol Chem 1996, 271:18253-62).

Gene expression was examined by PCR in cDNA samples as previously described Vu 1996, ibid.; Yao et al, J Clin Invest 2003, 111:265-73). cDNA samples were amplified in a 3.0 μL reaction mixture in the presence of 50 μM dNTP, 1 nM primer, 0.125 U KT1 DNA polymerase (Hu et al, J Biol Chem, 1997, 272-20715-20; Yao, 2003, ibid). The cDNAs and primers were heated to 95° C. for 1.5 min, then amplified by 35 cycles at 95° C. for 15 sec, 65° C. for 40 sec and 72° C. for 30 sec. PCR products underwent electrophoresis on 5% polyacrylamide-urea gel and scanned by phosphoImage Scanner (Molecular Dynamics, Sunnyvale, Calif.). The following PCR primers were used for mRNA quantitation:

Oct4:

(SEQ ID NO: 1)

5′-primer (#3284)-AGCACGAGTGGAAAGCAACTCAGA

(SEQ ID NO: 2)

3′-primer (#3285)-CTTCTGCAGGGCTTTCATGTCCTG
Ndp52L1:

(SEQ ID NO: 3)

5′-primer (#3288)-TAGAAGAGATGGAACAGCTCAGTGA

(SEQ ID NO: 4)

3′-primer (#3289)-ATTGACCCTCTGTGTTGCTTCCAGT
Dppa3:

(SEQ ID NO: 5)

5′-primer (#3290)-CTATAGCAAAGATGAGAAGACTTGT

(SEQ ID NO: 6)

3′-primer (#3291)-TGCAGAGACATCTGAATGGCTCACT
β-actin:

(SEQ ID NO: 7)

5′-primer (#1483)-TGAGCTGCGTGTGGCTCCCGA

(SEQ ID NO: 8)

3′-primer (#1484)-GATAGCACAGCCTGGATAGCA

FIG. 5 shows lanes 1 and 14 for 100 by DNA markers. Lanes 2, 6, 10 and 15 contain results from un-rejuvenated fibroblast control cells. Lanes 3, 7, 11 and 15 contain results from early ESL novacells. Lanes 4, 8, 12 and 17 contain results from novacell colonies. Lanes 5, 9, 13 and 18 show the results from novacells grown on feeder cells. The control fibroblasts were terminally differentiated and did not express the three ESC markers (Oct-4, Ndp52L1 and Dppa3). However, all three stages of the rejuvenated novacells expressed high levels of the three ESC markers. The internal control β-actin was equally expressed in all cell types, including controls. These data demonstrate that the in vitro rejuvenation method was able to dedifferentiate somatic cells into the ESL novacells that are pluripotent and capable of differentiating into other cells and tissues useful in replacement of ESCs in cell therapy.

### Example 10

### Formation of Pluripotent Cells by In Vitro Epigenetic Reprogramming

As in the preceding example, fibroblasts were trypsinized, and approximately 10⁶ cells were aliquoted into 1.5 mL tubes. The tubes were centrifuged and the supernatant poured off. Then 50 μL of trypsin-EDTA (Invitrogen) was added and the mixture was incubated for 5 min at 37° C. to permeabilize the cell membranes. The cells were spun at 1200 rpm for 3 min to pellet the cells. The cells were washed with Buffer T (20 mM HEPES, pH 7.3, 110 mM KAc, 5 mM NaAc, 2 mM MgAc, 1 mM EGTA, 2 mM DTT, 1 μg/mL each of aprotinin, pepstatin A, and leupeptin). Next the cells were resuspended in 20 μL rejuvenating buffer (2 mg/mL BSA, 2 mM ATP, 10 mM phosphocreatine, 40 U/mL creatine kinase, 0.5 μL RNase inhibitor, 5 μL Buffer T, 10 μL ES or embryo extract) and were rejuvenated 1 hr at 37° C. At the end of the rejuvenation period, to the rejuvenation solution was added 280 μL of KO-DMEM supplemented with 10% FBS, 1× of antibiotics (100 U/mL of penicillin and 100 μg/mL of streptomycin), 1 mM glutamine, 1% non-essential amino acids, 0.1 mM β-mercaptoethanol, 4 ng/mL basic fibroblast growth factor (bFGF), 0.12 ng/ml TGF-β and 10 ng/ml leukemia inhibitory factor (LIF).

As in Example 6, detergents (e.g., digitonin), streptolysin 0 (STO) or electroporation can also be used to pretreat the cell membrane before cell rejuvenation.

After rejuvenation, the cells do not automatically dedifferentiate into pluripotent ESL when they are directly plated. However, the rejuvenated cells have a short doubling time and other improved physiological functions. The following three-step selection process was used to isolate completely reprogrammed cells.

Rejuvenated cells were grown in 20 μL droplets. Cell droplets were placed on the cover of a 100 mm plate, which was then carefully inverted. PBS was placed in the bottom of the plate. The cells were incubated overnight. Rejuvenated cells were highly mobile at this stage; most moved upward to attach to the uncoated cover of the plate. To detach the cells from the cover, the cells were typsinized. All cell droplets were combined, to which was added 1 mL KO-DMEM. The cells were next grown on feeder cells (unrejuvenated cells or un-reprogrammed cells). The rejuvenated cells were maintained with daily medium changes. Aggregated ESL cell colonies are selected and grown as suspended cells in KO-DMEM supplemented with 4 ng/mL bFGF, 0.12 ng/mL TGF-β1 and 10 ng/mL LIF on top of 0.5% agarose gel. The unreprogrammed and partially reprogrammed cells did not survive in the suspension medium. After several passages (2-4 d), the aggregated ESL novacells were transferred to grow on the feeder cell layer. Cells with similar morphology of ESCs and healthy cell types were selected to expand for further analysis of ES markers. After several passages, these cells were saved in liquid nitrogen or used for cell typing and differentiation assay.

After these three steps of special treatments (inverted droplets, suspended cells on agarose and ESC selection), these cultured novacells had different cell morphology from the original aged cells. These novacells are pluripotent and can replace normal heterogeneous ESCs in cell therapy. Because these cells came from the recipient, the autogolous novacells do not cause graft-versus-host reactions that can occur with umbilical cord blood stem cells and other cellular transplants.

### Example 11

### Converting Human Skin Fibroblasts into Pluripotent Novacells by In Vitro Epigenetic Reprogramming

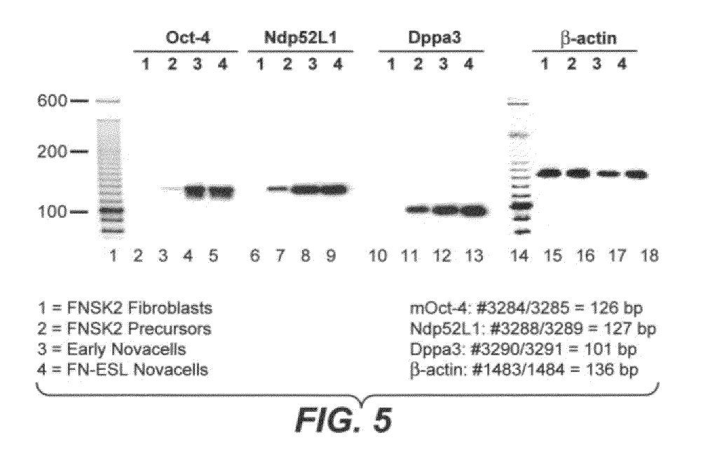

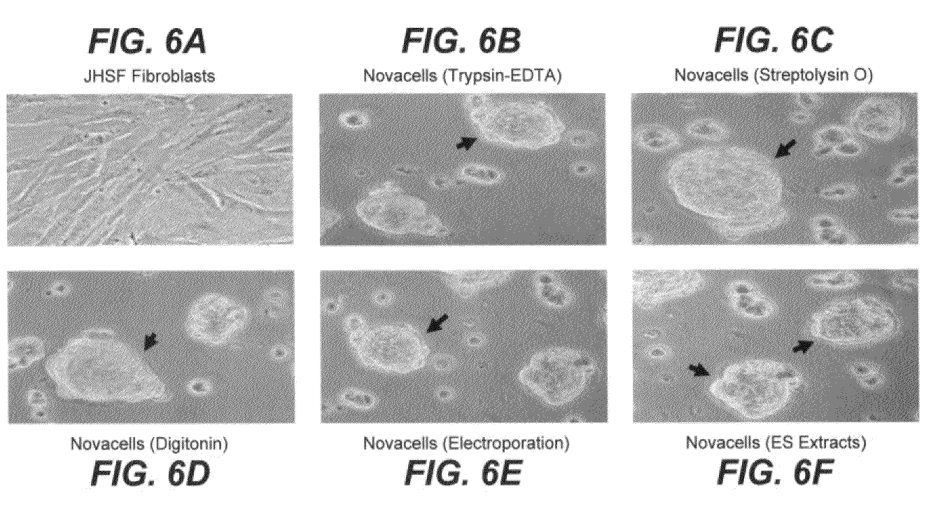

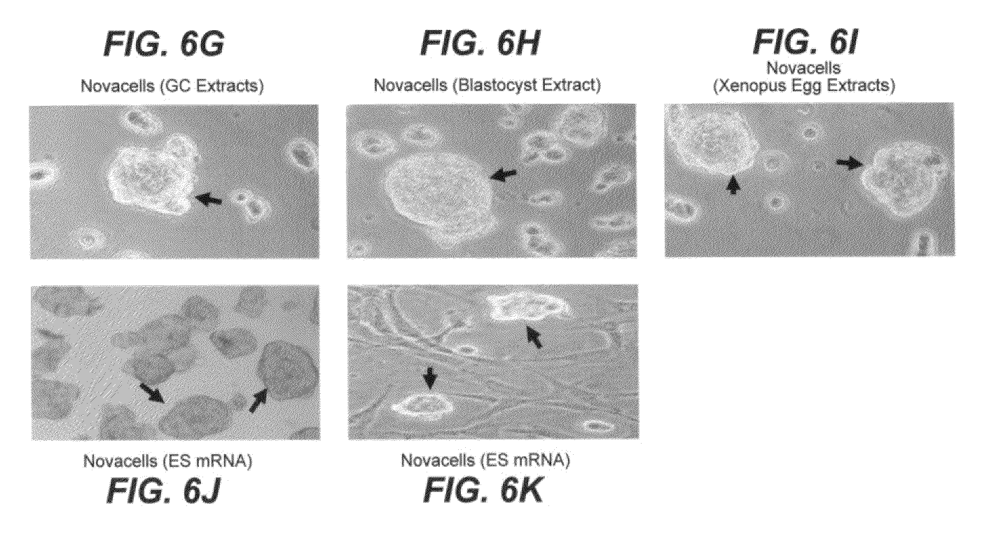

Fibroblasts were cultured from a 49-yr-old human male and were rejuvenated in vitro with different pre-treatments and different cell extracts. After rejuvenation, novacells were selected to grow on top of agarose gel and photographs of novacell colonies were taken under microscope. Data clearly showed that pretreatment of fibroblasts with trypsin-EDTA (FIG. 6B), streptolysin 0 (FIG. 6C), digitonin (FIG. 6D) and electroporation (FIG. 6E) yielded similarly rejuvenated cells, compared to the untreated control fibroblasts (FIG. 6A). Furthermore, fibroblasts were similarly rejuvenated by extracts of ESC nuclei (FIG. 6F), GC cells (FIG. 6G), blastocysts (FIG. 6H) and Xenopus eggs (FIG. 6I).

### Example 12

### Formation of Pluripotent Novacells by Replication-Defect ESC Fusion

Several research groups have reported forming ESCs from somatic cells (e.g., fibroblasts) by in vitro hybridization or fusion with ESCs (e.g., Tada et al, Current Biology 2001, 11:1553-58; and Cowan et al, Science 2005, 309:1369-73). However, the formed ESCs are tetraploid cells containing the genome of both the target cell and ESCs. Tetraploid cells are undesirable for clinical applications as they are believed to be genetically unstable.

To solve this problem, a novel approach was developed to create diploid, not tetraploid, pluripotent ESCs from somatic cells by using an “ESC replication-defect” (ESR) method. In this method, we first disabled the DNA replication machinery of the reprogramming ESCs so that the genome of ESCs does not replicate or contribute to the newly joined cells of the fusion. Although the ESC replication is inactivated, for a little while, the existing genome still produces mRNA and subsequently proteins of reprogramming factors that are essential in trans-differentiating the target cell into ESCs. The resulting pluripotent ESCs were diploid. Most importantly, these are individualized ESC and are thus useful in replacing ESCs in treatment of diseases.

The methods used to disable DNA replication include, but are not limited to, the exposure of the ESCs to radiation, chemical compounds, chemotherapeutics, viral and physical treatment. These methods have been used to block DNA replication of feeder cells. Two methods of disabling DNA replication were tested in ESCs.

In one method, ESCs were exposed to radiation (Cs, 3000 rds). After exposure, ESC DNA was damaged and would not replicate to produce daughter cells. However, these treated ESCs survived in culture up to about one week, and many genes, including those required for cell reprogramming, were still active and expressing protein. As a result, the irradiated ESCs can be used as a reliable source to provide reprogramming factors to convert somatic cells into pluripotent ESCs. Due to the replication-defect feature, the genome of the ESC did not replicate and thus did not contribute to the daughter cells' genome after cell fusion.

In another method, DNA replication was inactivated by exposing ESCs to 0.5 μg/mL actinomycin D overnight. After treatment, actinomycin D blocked DNA replication in ESCs but keeps protein synthesis machinery intact. After cell fusion, the treated ESCs contributed reprogramming factors in fusion cells but did not contribute to the daughter cells' genome. After several passages of selection, only those diploid ESCs grew and were available for therapeutic applications.

For cell fusion, equal amounts of the replication-defect ESCs and the target somatic cells (e.g., fibroblasts) were mixed and washed twice with CMF buffer (calcium- and magnesium-free HBSS). The cell pellet was centrifuged to completely remove the remaining buffer. Then the tube was tapped to loosen and mix the cell pellet, after which 1.5-2 mL PEG (polyethylene glycol 1500, Cat. No. 783641, Roche, Germany) was added over 1 min. To mix the cells and PEG, the tube was again tapped and rotated. Next, 20 mL of pre-warmed (37° C.) PMF buffer (0.2 M PIPES, pH 6.95, 2 mM MgSO₄ and 4 mM EGTA) was added dropwise over 3-5 min, without breaking up cell clumps. An additional 20 mL of PMF buffer was slowly added and the tube carefully inverted to mix the cells. Then the cells were centrifuged at 1200 rpm for 5 min and resuspended with KO-DMEM medium supplemented with 4 ng/mL bFGF and 0.12 ng/mL TGF-β1. As described above, the fused cells were cultured in suspension on top of agarose gel or on gelatin-coated plates.

Alternatively, electroporation and virus-mediated cell fusion can be used to create fusion cells from replication-defect ESCs and the target cells. For electroporation, equal amounts of replication-defect ESCs and target somatic cells were mixed and washed 3 times in PBS. The cells were suspended in 0.3 M mannitol buffer at a concentration of 10⁶ cells/mL. Hybrid cells were produced by electric fusion (E=2.5-3.0 KF/cm) using BTX Electro Cell Manipulator 2000 with slide glasses carrying a 1-mm electrode gap (BTX, Holliston, Mass.). After fusion, cells were cultured and selected in KO-DMEM medium supplemented with 4 ng/mL bFGF and 0.12 ng/mL TGF-β1.

After fusion, the nuclei of the somatic cells were epigenetically reprogrammed in the fused cells by the factors provided by the replication-defect ESCs. After several passages, the genome of the original replication-defect ESCs had completely disappeared and left the reprogrammed somatic genome in the fusion cells. After selection, the cultured new cells possessed pluripotency and were diploid cells, thus useful in cell replacement therapy.

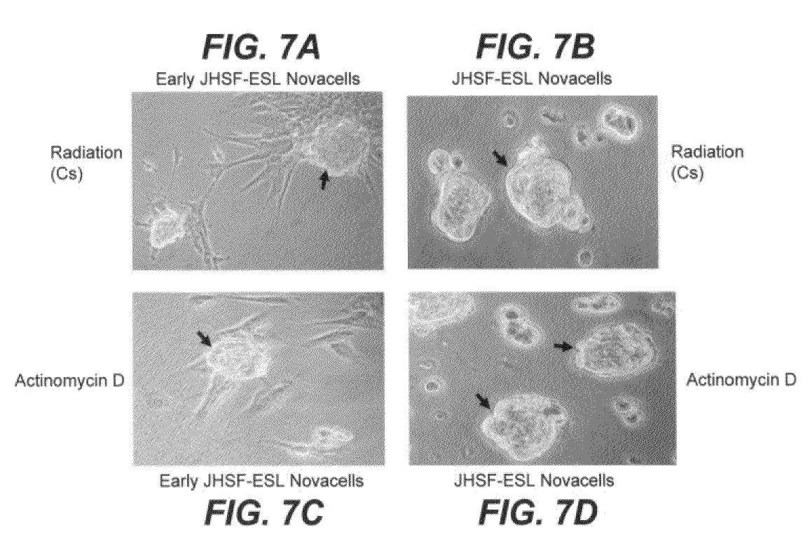

Data showed that both radiation (FIGS. 7A and 7B) and actinomycin D (FIGS. 7C and 7D) caused defective DNA replication. After cell fusion, diploid ESL-novacells were selected and expanded. These diploid ESL-novacells were ESCs from the patients and did not have the risk of contamination of the genome of the reprogramming donor cells (E12 ESCs). These fused cells had the same morphology and growth rate as ESCs. Similarly the fused cells expressed the ESC biomarkers Oct4, Nanog and Stellar. Thus, the fused cells are useful for cell replacement therapy.

### Example 13

### Differentiation of Pluripotent Novacells

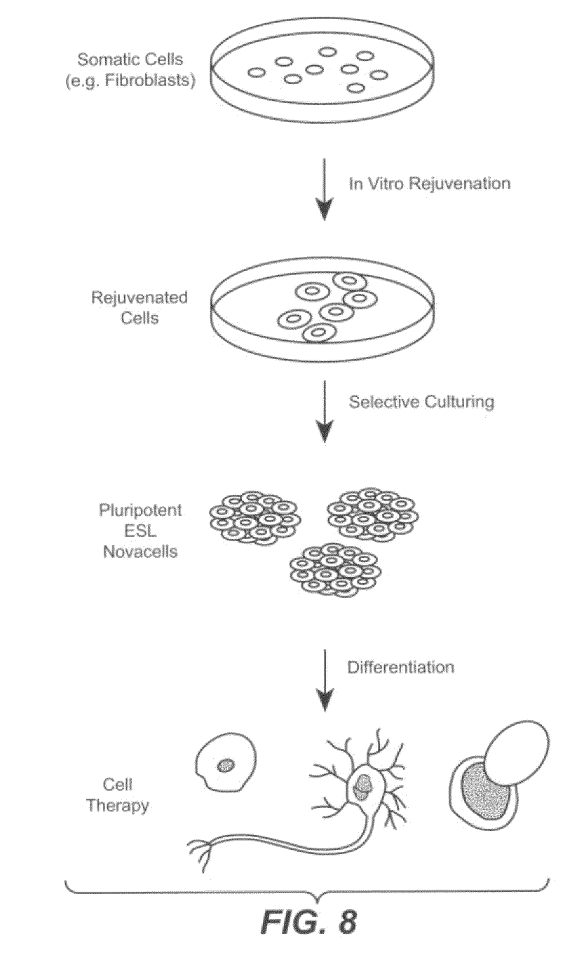

FIG. 8 is a schematic outline of the inventive procedure to rejuvenate aged cells into pluripotent novacells and then into differentiated cells. Like FIG. 1, aged cells were first collected and cultured in appropriate media to expand the cell population. After exposing the cells to a cell membrane permeabilizing reagent (e.g., trypsin/EDTA) to open the gap junctions of the cell, cells were rejuvenated with the rejuvenating factors in the rejuvenating buffer. After rejuvenation, cells were grown in appropriate media supplemented with specific growth factors. The colonies of pluripotent novacells were then selected on feeder cells or coated matrix (e.g., matrigel or agarose). The selected novacells have the basic features of embryonic stem cells (ESCs) or other tissue-specific stem cells and are useful to replace ESCs and stem cells in cell therapy. One of the advantages of these novacells is that they can be derived from the same patient and thus significantly reduce or eliminate the chance of immune rejection on return to the patient. Another advantage is that there are no ethical or political concerns when novacells are used in cell therapy because the use of human embryos is avoided.

These methods vary with the type of cells sought. In general, published methods used to differentiate embryonic stem cells are suitable for differentiating ESL-novacells. Following are examples of how novacells differentiated into adipocytes and osteocytes.

ESL novacells were cultured in KO-DMEM medium with 20% FBS at 37° C. in the presence of 1000 U/mL of LIF. Cell suspensions were made into hanging droplets (30 μL) onto the cover of a Petri dish. The bottom of the dish was filled with PBS. After 2 d, the embryoid bodies (EBs) were formed and collected onto a fresh culture dish pre-coated with 0.1% gelatin. For adipocyte differentiation, the attached EBs were treated with 10⁻⁶ M of all-trans-retinoic acid (ATRA) for 3 d followed by 10⁻⁷ M of insulin and 2×10⁻⁹ M of triiodothyronine (T3). For osteoblast differentiation, EBs were treated with 10⁻⁸ M of 1.25(OH)₂ vitamin D₃ starting at the 5^{th} day in medium containing 3×10⁻⁴ M of ascorbic acid phosphate and 10⁻² M γ-glycerophosphate. Cultures were terminated at days 10, 20, and 30 and used for immunohistochemical staining. After differentiation, formation of adipocytes and osteocytes was confirmed by immunohistochemical staining.

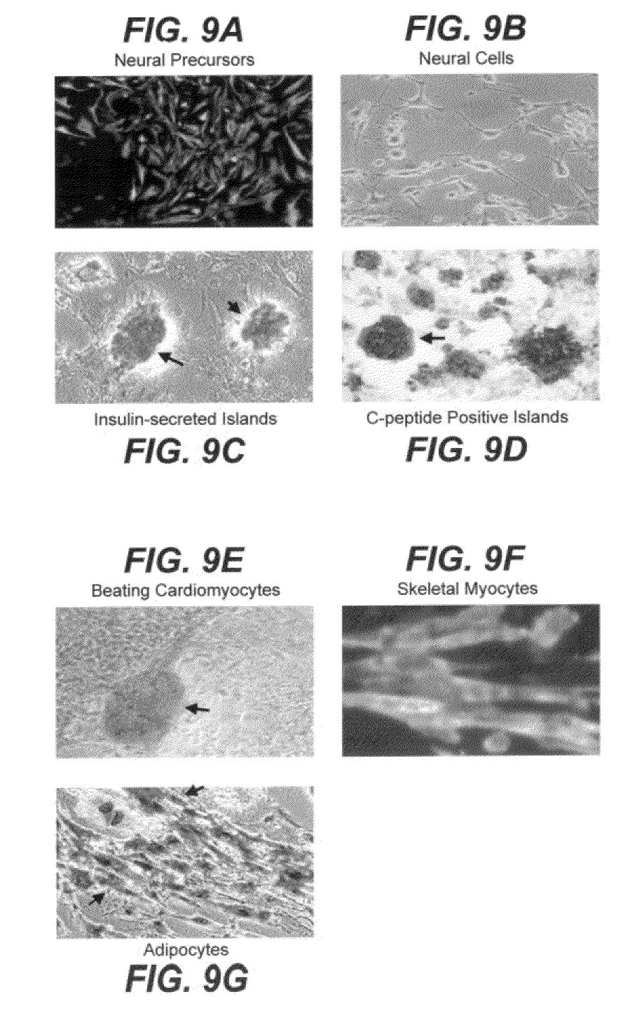

For staining the lipid in differentiated adipocytes, cells were washed with PBS and were fixed in 10% neutral formalin for 2 min at room temperature. After rinsing with tap water, cells were stained with Oil-Red-O for 10-12 min until the oil drops were visibly stained under microscope. Slides were then rinsed with 50% isopropanol and tap water. Cells were counterstained with hematoxylin for 10 min. Differentiated adipocytes were observed under optical microscopy (FIG. 9G). There was no lipid staining in untreated control fibroblasts (not shown). In contrast, after differentiation, FN-ESL novacells synthesized lipids accumulated in the cytoplasm. These data indicate that FN-ESL novacells had the potential to and did differentiate into adipocytes.

### Example 14

### Differentiation of FN-ESL Novacells into Skeletal Myocytes

The EBs were formed as described above. FN-ESL cells therefrom (approximately 5×10⁵) were transferred into an inverted 10 mm bacteriological Petri dishes in Iscove's Modified Eagle Medium (Invitrogen) supplemented with 20% FBS, 2 mM L-glutamine, 1× nonessential amino acids, 450 μM monothioglycerol (Sigma) and antibiotics. After 5-7 d, EBs were plated onto 0.1% gelatin-coated 6-well tissue culture dish at a density of 7-10 EBs per well and were cultured for 4 d. Cells were washed with Dulbeccos-PBS (D-PBS), and were incubated with 2 mL/well of DMEM supplemented with 2% inactivated horse serum and 1 mL of supernatant of C1C12 culture medium (filtered by a 0.2 μm filter). Medium was changed daily for 2-4 days. Myotube formation during the differentiation was followed by microscopic observation.

Differentiation of skeletal muscle was confirmed by immunohistochemical staining Differentiated cells were washed with D-PBS 3 times and were fixed with 100% ethanol for 5 min. Background was blocked with 1-2% normal goat serum in D-PBS containing 0.05-0.1% saponin for 1 hr at room temperature. The primary antibody was diluted in D-PBS containing 4 mg/mL of BSA and 0.05-0.1% saponin. Mouse anti-MHC primary antibody (Sigma, 1:500) was added and incubated at room temperature for 1-3 hr or overnight at 4° C. Cells were then washed with D-PBS 5 times (2 for a quick rinse, 1 for 15 min, and 2 for 5 min). The second antibody solution (goat anti-mouse 1:1000) was added and incubated at room temperature for 0.5-1 hr. After washing 5 times (same protocol) with D-PBS containing 0.05% saponin, the cells were viewed by Zeiss Axiovert 200 inverted fluoresce microscopy.

There was no immunostaining of skeletal muscle protein in control cells (not shown). After differentiation, FN-ESL novacells were aggregated and fused into myotubes and synthesized skeletal muscle-specific proteins (FIG. 9F). These data show that FN-ESL novacells had the potential to and did differentiate into skeletal muscles.

### Example 15

### Differentiation of FN-ESL Novacells into Cardiomyocytes

FN-ESL novacells were cultured on BMM2/NG feeder cells in Knockout Dulbecco's modified Eagle Medium (KO-DMEM) supplemented with 20% FBS, 1000 U/mL LIF, L-glutamine, nonessential amino acids and β-mercaptoethanol. BMM2/NG feeder cells were pretreated by γ-irradiation at 30 Gray to stop their replication. FN-ESL novacells were seeded in Petri bacterial culture dishes at a density of approximately 2.0×10⁶ cells in KO-DMEM in the absence of LIF to form EBs. After 3 d, EBs were collected and plated onto 1% Matrigel-coated tissue culture dishes in 10% FBS/KO-DMEM. Two hr later, 100 ng/mL activin A was added to the medium and the cells cultured for 24 hr. Medium was replaced with 10% FBS/KO-DMEM again for 6 hr. Then 10⁻⁶ M ATRA was added to the medium and the cells cultured for another 24 hr. Cells were treated with 10% FBS/KO-DMEM with 10 ng/mL bFGF for 3 d and were switched to N2 medium containing DMEM/F12 (1:1) supplemented with B27, 1 μg/mL of laminin, 10 mM nicotinamide and 10 ng/mL bFGF. This N2 medium was changed daily until analysis.

At the 4^{th} day in N2 medium, there were beating cell clusters in the culture. Some of the beating cardiomyocytes were transferred to slides and were fixed by 4% paraformaldehyde in PBS overnight at 4° C. and then washed with phosphate buffer twice. Non-specific binding-sites were blocked with horse serum for 1 hr at room temperature. Incubations with the primary and secondary antibodies were 1 hr at room temperature. The following primary antibodies and dilutions were used: insulin AB-6 mouse monoclonal antibody (Lab Vision, Fremont, Calif.) 1:200, Troponin T mouse monoclonal antibody (Lab Vision, Fremont, Calif.) 1:100 and anti-C-Peptide antibody (LINCO Research, Inc., St. Louis, Mo.) 1:100. Secondary ready-to-use universal antibody was applied according to the manufacturer's instructions. DAB (3,3′-diaminobenzidine) was used as the reaction substrate. Images were captured by Zeiss Axiovert 200 inverted microscope (FIGS. 9C-E).

These data show that FN-ESL novacells were able to differentiate into functional beating cardiomyocytes.

### Example 16

### Differentiation of FN-ESL Novacells into Insulin-Secreting Pancreatic Beta Cells

The EBs were formed as described above. A three-step method as described for mouse ESC (Shi et al, Stem Cells, 2005, 23:656-62) was used with minor modifications to differentiate insulin-secreting cells. A standard immunohistochemistry protocol was carried using ready-to-use Vectastain universal quick kit (Vector Laboratories, Inc., Burlingame, Calif.). Briefly, cells were fixed by 4% paraformaldehyde in PBS overnight at 4° C. and then washed with PBS twice. Non-specific binding sites were blocked with horse serum after which the cells were incubated with insulin Ab-6 mouse monoclonal antibody (1:200; Lab Vision) and anti-D-Peptide antibody (1:100; Linco Research, Inc.). Secondary ready-to-use universal antibody was applied according to the manufacturer's instructions. DAB was used as the reaction substrate. Images were captured by Zeiss inverted microscope. There was no immunostaining of insulin in control cells. After differentiation, some FN-ESL novacells were observed to have aggregated into cell islands. Cells in the islands synthesized insulin visible in their cytoplasm (brown in FIGS. 9C and 9D). The longer induction led to accumulation of insulin signals in the big mass of cells. These data show that FN-ESL novacells had the potential to and did differentiate into insulin-producing cells.

### Example 17

### Differentiation of FN-ESL Novacells into Neural Cells

Generation of neuroectodermal cells from FN-ESL novacells was performed by the method described previously by Zhang et al. (2001, Nat Biotechnol 19:1129-33). Briefly, upon aggregation to EBs, differentiating ESCs formed large numbers of neural tube-like structures in the presence of FGF-2. Neural precursors were isolated and purified on the basis of their differential adhesion. Following the replacement of FGF-2 with brain-derived neurotrophic factor (BDNF), the cells differentiated into neurons, astrocytes, and oligodendrocytes. Immunohistochemical staining of neural cells was performed as previously described (Zhang ibid.). Primary antibodies used in this study included polyclonal antibodies against nestin (Chemicon, Temecula, Calif., 1:750) and βIII-tubulin (Covance Research Products, Berkeley, Calif., 1:2000). Antigens were visualized using appropriate fluorescent secondary antibodies (FIGS. 9A and 9B). These data show that ESL novacells were able to and did differentiate into neural cells.

### Example 18

### Method of Converting Human Wilms' Tumor Cell Line into ESL Novacells

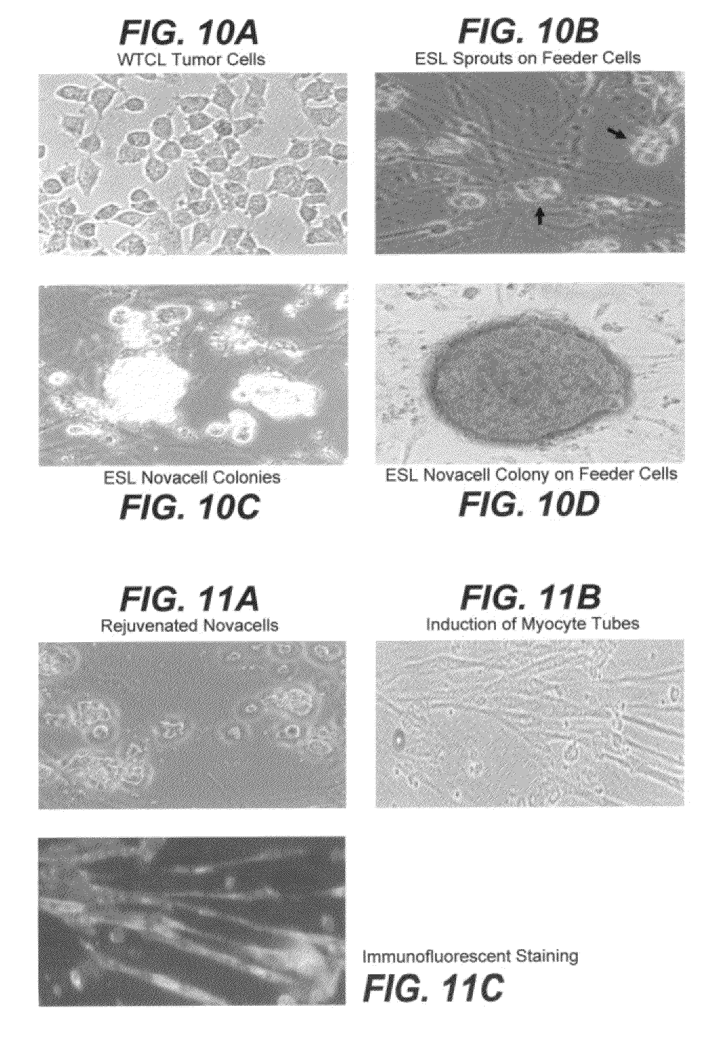

To illustrate the conversion of human cells into nova cells, a human Wilms' tumor cell line (WTCL) was rejuvenated in vitro by the nuclear extracts of the mouse ESC using the method described above. The rejuvenated cells were cultured and selected on embryonic fibroblast feeder cells. After culturing, some cells became aggregated and formed small novacell colonies with ESC morphology. The cell colonies were carefully picked up and seeded on feeder cells. FIG. 10A shows unchanged WTCL tumor cells. FIG. 10B shows WT-ESL novacell colonies. FIG. 10C shows WT-ESL sprouts on feeder cells; and FIG. 10D shows a WT-ESL novacell colony on feeder cells. These results show that the in vitro rejuvenation method was able to induce cell dedifferentiation of human cells into embryonic stem-like cells. These WT-ESL novacells then can be differentiated into various cell types using the methods described above. After rejuvenation, tumor cells showed less or no tumor-producing capacity as shown by fewer agar-gel-forming colonies and no tumors in nude mice. This rejuvenation-induced dedifferentiation may provide a breakthrough strategy to develop tumor therapies.

### Example 19

### One-Step Rejuvenation and Differentiation (OSRD) Protocol

As described above, the aged somatic cells were first rejuvenated into pluripotent novacells and subsequently differentiated into other cells, like adipocytes, osteocytes, cardiocytes, skeletal muscle, and insulin-secreting cells. These procedures may take months. To speed up the processes, we combined these two procedures into a simple one-step protocol (hereinafter the OSRD procedure) using ESC nuclear extracts as the rejuvenating factors and specific cell extracts as the differentiation inducer. Following is an example of starting with fibroblasts and ending with skeletal muscle. Fibroblasts were treated simultaneously with the nuclear extracts of ESCs and muscle extracts.

Fibroblasts (approximately 10⁶ cells) were aliquoted in 1.5 tubes. After treatment with 50 μL trypsin-EDTA (Invitrogen) and washing with Buffer T, the cells were resuspended in 50 μL rejuvenating buffer (1 ng/mL BSA, 1 mM ATP, 5 mM phosphocreatine, 25 μg/mL creatine kinase (Sigma), 2 U RNasin [Promega, Madison, Wis.], 100 μM GTP, and 1 mM dNTPs (nucleotides triphosphate), containing ESC nuclear extracts and skeletal muscle extracts. The cells were rejuvenated at 37° C. for 1 hr. Then the processed cells were grown in inverted drops in KO-DMEM supplemented with 20% FBS, 1× antibiotics (100 U/mL penicillin and 100 μg/mL streptomycin), 1 mM glutamine, 1% non-essential amino acids, 0.1 mM β-mercaptoethanol, 4 ng/mL bFGF, 0.12 ng/mL TGF-β1 and 10 ng/mL LIF. During the morning of day 2, the inverted drops were collected and combined, and the collected cells were grown on gelatin-coated plates in DMEM supplemented with 2% inactivated horse serum and 1 mL of supernatant of murine myoblast cell (C1C12) culture medium (filtered by 0.2 μm filter). Medium was changed daily for 2-4 days. Myotubes formed during the differentiation were examined by immunofluorescent staining.

Formation of skeletal muscle was examined by microscopic photophotographs and immunohistochemical staining. After rejuvenation, fibroblasts grew out as the small EBs on agarose gel (FIG. 11A). At differentiation, cells were aggregated and fused into myotubes (FIG. 11B) and synthesized skeletal muscle-specific proteins (FIG. 11C). These data show that using one-step rejuvenation differentiation, fibroblasts can be directly rejuvenated and differentiated into skeletal muscle.

### Example 20

### Reactivation of Telomerase

True germ cells and stem cells contain an enzyme called telomerase that replaces telomeres, thus preventing them from experiencing the Hayflick Limit. In human germ cells, and approximately 85% of cancer cells, the enzyme human TElomerase Reverse Transcriptase (hTERT) and an RNA template are sufficient to create new telomeres.

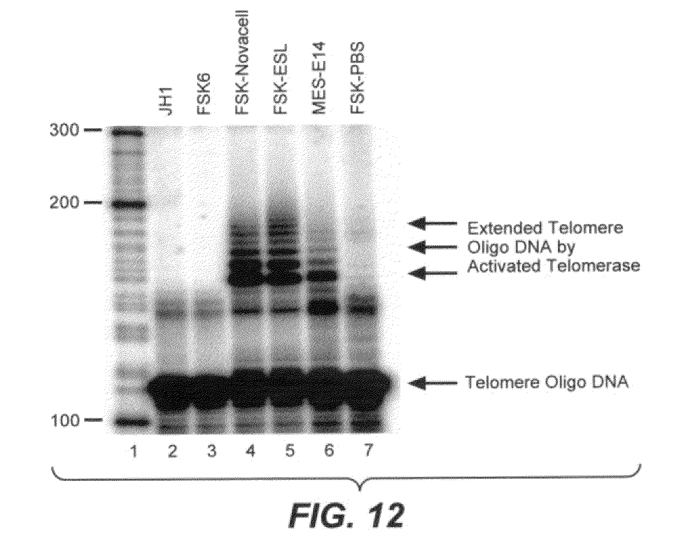

To determine whether the rejuvenated cells described above share the presence of telomerase with germ and stem cells, we measured telomerase activity by the TRAP assay (Kim N W et al, Science 1994 266:2011-15). FIG. 12 shows telomerase products for unrejuvenated and rejuvenated fibroblasts. Shown in lanes 2 and 3, including human skin JH1 fibroblasts and mouse skin FNSK6 fibroblasts, respectively, the unrejuvenated cells have no detectable products of telomerase. Like the ESC results shown in lane 6, two types of rejuvenated cells produced the products of telomerase (lanes 4 and 5), evidence of the activity of telomerase in the cells.

### Example 21

### In Vivo Methods of Rejuvenating Tissue or Organs

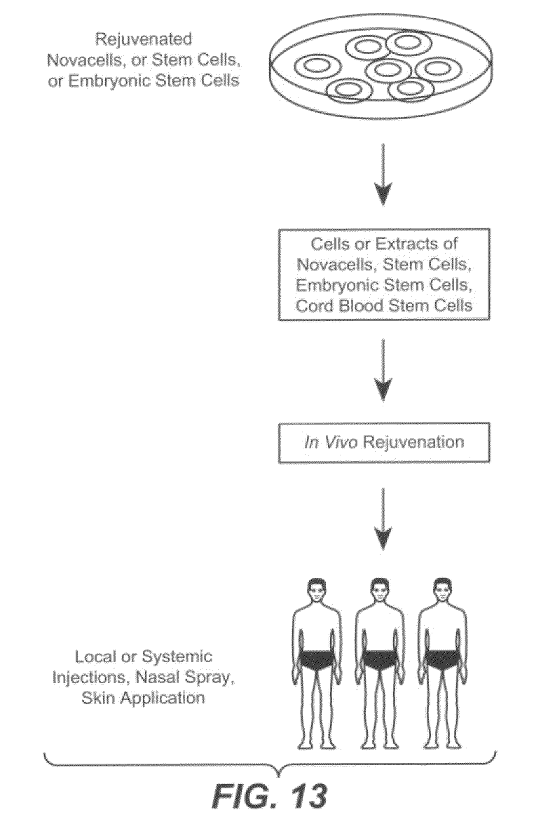

Rejuvenating factors mentioned supra, including nuclear extracts, cell extracts of embryonic stem cells, stem cells, cord blood stem cells and nova cells, can also be used directly in in vivo rejuvenation methods to rejuvenate tissues, organs and the body. This can be done by locally applying the rejuvenating factors systemically or by injecting the rejuvenating factors to the desired site of action. This is summarized in FIG. 13.

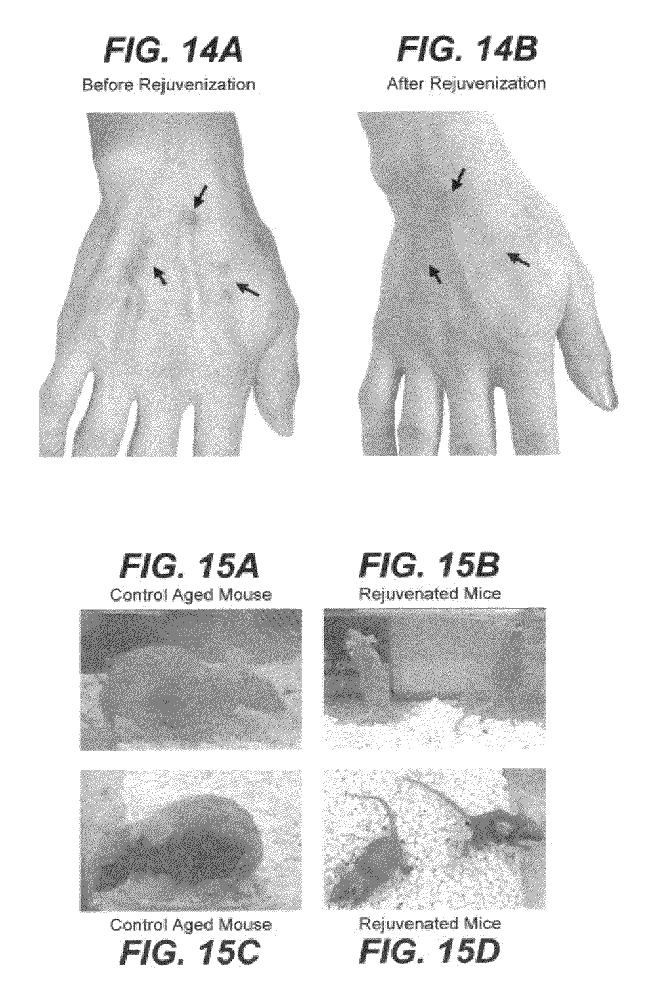

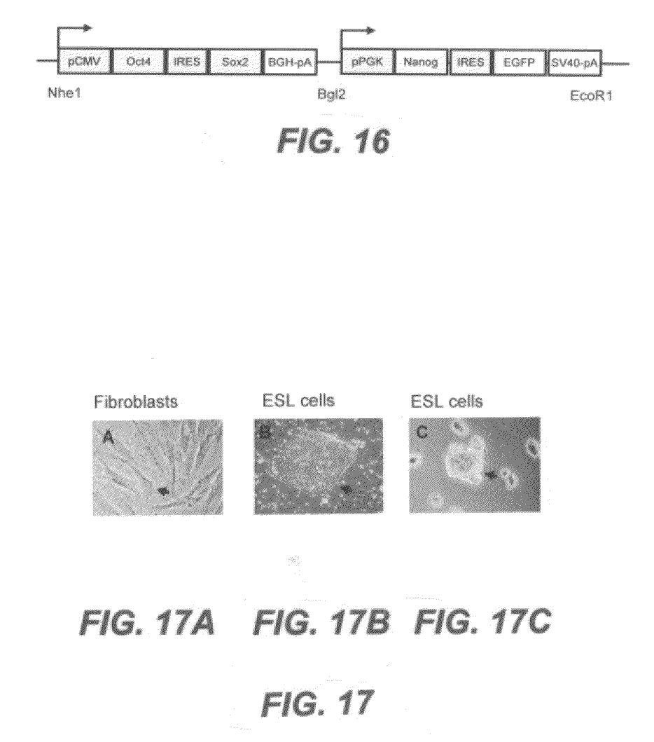

As an example of in vivo rejuvenation, we tested the removal of skin pigmentation in a male volunteer, who had several injury-caused pigmented areas on his right hand. The skin can be rejuvenated (e.g., reducing pigmentation and wrinkles) with nuclear extracts or cell extracts of ES cells locally applied to the skin. The area to be rejuvenated was first sterilized with 70% isopropyl alcohol. A layer of trypsin-EDTA (Invitrogen) was then applied to the area and maintained for 10 min without drying. The skin was then washed with 0.9% saline solution. Two layers of ALL-Gauze-sponge were soaked in human ESC nuclear extracts prepared in 1× rejuvenating buffer and gently applied to the permeabilized area. To prevent evaporation, the ES-extract-soaked sponge was covered by thin plastic and the edges of the plastic were sealed to the skin with appropriate tape. After overnight rejuvenation, the skin was washed with 0.9% saline and a thin layer of skin lotion was applied to the rejuvenated area. This procedure was repeated once or twice a week for 2 wk and can be repeated as necessary. FIG. 14A shows the pigmented skin area (arrows) before treatment. FIG. 14B shows the much less pigmented area after 2 wk of treatment. After rejuvenation, the skin became smooth, shiny and fresh. The pigmentation also completely disappeared after the rejuvenating treatment. The patient experienced no discomfort. These data show that it is highly feasible to rejuvenate tissue in vivo.

Another method to rejuvenate the skin is to subcutaneously inject the nuclear extracts or cell extracts of ESCs and stem cells under the skin twice a week. The factors in the nuclear extracts rejuvenate skin cells and remove pigmentation and wrinkles. Yet another method to rejuvenate the skin is to subcutaneously inject ESCs, stem cells or cord blood stem cells under the skin. The injected cells multiply under the skin and secrete growth factors that rejuvenate skin cells and remove pigmentation and wrinkles.

### Example 22

### Method of Rejuvenating the Whole Body

The rejuvenating factors disclosed above can be delivered systemically to rejuvenate the whole body. The rejuvenating factors include, but are not limited to, nuclear extracts and cell extracts derived from ESCs, stem cells, cord blood stem cells and novacells. The cultured cells, including stem cells, cord blood stem cells and novacells can also be used for this purpose. To rejuvenate the body, the nuclear extracts or cell extracts of ESCs, stem cells, cord blood stem cells and novacells can be directly administered to the human body using recognized and practical clinical methods, including intravenous injection, subcutaneous injection, intramuscular injection, intrathecal injection, nasal spray, implantation of slow-release pellets, topical application, etc. Rejuvenating factors in nuclear extracts or cell extracts are exposed to every tissue and organ of the body. Cells, including ESC, stem cells, cord blood stem cells and novacells also can be administered using routine methods. These cells are capable of surviving and multiplying when they reach tissues and organs. Locally, they will be differentiated and replace the aged cells.

In one study, rejuvenating agents were systemically used to rejuvenate animals. Old athymic mice (nu+/nu+, 2 yr old) were divided into three groups. Group 1 (2 mice) received ESC extracts by tail vein, 1 mL extract/mouse, twice weekly for a total of 3 wk. Group 2 (2 mice) received PBS control solution by vein. Group 3 (2 mice) received GN-ESL novacells by tail vein (about 10⁷ in 1 mL) twice weekly for 3 wk. Food consumption and body weight were recorded every two days. Animal activities were recorded by camera.

FIGS. 15A and 15C show PBS-control aged mice. FIG. 15B shows mice rejuvenated with ESC extracts. FIG. 15D shows mice rejuvenated with novacells. After 3 wk of treatment, the control group experienced no significant changes in all measured variables, including body weight, food consumption, appearance and activity. However, mice treated with the ESC extract or with FN-ESL novacells consumed more food, although there were no significant differences in body weight. At the same time, the rejuvenated mice were more physically active and more energetic than control mice. Most interestingly, the thin wrinkled skin appeared smoother, thicker and healthier on rejuvenated animals than on control animals. These data, although preliminary, suggest that rejuvenation by ESC extract or FN-ESL novacells improves the life of aged animals.

These in vivo rejuvenation methods can be used to enhance immune functions, improve the general body health, increase the capacity for sports, help disease and paralysis recovery, prolong a person's lifespan, correct congenital defects of CNS system, repair injured or aged organs (e.g., heart, kidney, liver and brain), turn aged white hair into youthful black hair, reduce skin wrinkles and pigmentation, and treat neurodegenerative diseases like Alzheimer's Disease, Parkinson's disease, amyotrophic lateral sclerosis (Lou Gehrig's disease) and stroke.

### Example 23

### Rejuvenation of Somatic Cells to Pluripotent Embryonic Stem-Like (ESL) Cells by a Two-Step In Vitro Cell Reprogramming

This invention has been described in an illustrative manner, and it is to be understood that the terminology used is intended to be in the nature of description, rather than limitation. Obviously, many modifications and variations of the present invention are possible in light of the above teachings and one of ordinary skill in the art, in light of this teaching, can generate additional embodiments and modifications without departing from the spirit of or exceeding the scope of the claims of this invention. Therefore, it is to be understood that within the scope of the appended claims, the invention may be practiced otherwise than as specifically described. Accordingly, it is to be understood that the drawings and descriptions herein are proffered by way of example to facilitate comprehension of the invention and should not be construed to limit the scope thereof.

1-51. (canceled)

52. A method of rejuvenating somatic cells, the method comprising the steps of
a. providing at least one human ES cell transcription factor in a mammalian expression vector;
b. transfecting the vector of step a into exponentially growing human cells;
c. after transfection, isolating the cells expressing the vector;
d. growing the isolated vector-expressing cells on plates until confluence;
e. collecting the vector-expressing cells;
f. treating the vector-expressing cells with membrane-permeabilizing solution and ES cell extracts;
g. sealing the membranes of the extract-treated cells; and
h. placing the extract-treated cells in hanging droplets to grow and isolating fully reprogrammed cells in clusters.

53. The method of claim 52, wherein the human ES cell transcription factors are Oct4, Sox3 and Nanog.

54. The method of claim 52, wherein the gene and its gene products of the human ES cell factors are introduced into the cell by electroporation.

55. The method of claim 53, wherein the factors are delivered into the cell by a cocktail of mixed gene products constructed in separate vectors.

56. The method of claim 53, wherein the factors are delivered into the cell by a single vector containing a tandem expression cassette of factors separated by the internal ribosome binding sequences (IRES).


SEQUENCE LISTING (8 sequences)

SEQ ID NO: 1 - DNA, 24 bp, Mus sp.
agcacgagtggaaagcaactcaga

SEQ ID NO: 2 - DNA, 24 bp, Mus sp.
cttctgcagggctttcatgtcctg

SEQ ID NO: 3 - DNA, 25 bp, Mus sp.
tagaagagatggaacagctcagtga

SEQ ID NO: 4 - DNA, 25 bp, Mus sp.
attgaccctctgtgttgcttccagt

SEQ ID NO: 5 - DNA, 25 bp, Mus sp.
ctatagcaaagatgagaagacttgt

SEQ ID NO: 6 - DNA, 25 bp, Mus sp.
tgcagagacatctgaatggctcact

SEQ ID NO: 7 - DNA, 21 bp, Mus sp.
tgagctgcgtgtggctcccga

SEQ ID NO: 8 - DNA, 21 bp, Mus sp.
gatagcacagcctggatagca